(19) United States
(12) Patent Application Publication
(10) Pub. No.: US 2026/0347462 A1
(43) Pub. Date: Nov. 5, 2026

(54) CALRETICULIN-TARGETED NANOBODIES, KITS, METHOD OF MAKING, AND METHODS OF USE THEREOF

(71) Applicant: THE UAB RESEARCH FOUNDATION, BIRMINGHAM, AL (US)

(72) Inventors: Benjamin M. LARIMER, BIRMINGHAM, AL (US); J. Bart ROSE, BIRMINGHAM, AL (US); Lucinda HALL, BIRMINGHAM, AL (US); Rachael GUENTER, BIRMINGHAM, AL (US); Mikhail PAVLENOK, BIRMINGHAM, AL (US); Michael NIEDERWEIS, BIRMINGHAM, AL (US); Logan MAVAR, BIRMINGHAM, AL (US)

(21) Appl. No.: 19/658,051

(22) Filed: Apr. 24, 2026

Related U.S. Application Data
(60) Provisional application No. 63/794,066, filed on Apr. 24, 2025.

(51) Int. Cl. C07K 16/28; A61K 49/00; A61K 51/10; A61P 35/00; G01N 33/575

(52) U.S. Cl. CPC C07K 16/28; A61K 49/0058; A61K 51/1096; A61P 35/00; G01N 33/5758; C07K 2317/569

(57) ABSTRACT

Described herein are calreticulin (CALR) targeting nanobodies, methods of using, methods of making, and kits comprising CALR-targeting nanobodies described herein. In examples, described herein are CALR-targeting nanobodies that target the extracellular component or region of CALR. In examples, described herein are probes comprising nanobodies described herein that can be used, for example, in imaging.

### CROSS-REFERENCE TO RELATED APPLICATIONS

This application claims the benefit of U.S. Provisional Application No. 63/794,066 filed on Apr. 24, 2025, which is incorporated herein by reference in its entirety as if fully set forth herein.

### SEQUENCE LISTING

The instant application contains a Sequence Listing which has been submitted electronically in .XML format and is hereby incorporated by reference in its entirety. Said .XML copy, created on Apr. 24, 2026, is named “222120_1100_Sequence_Listing.xml” and is 20,717 bytes in size.

### BACKGROUND

Nanobodies, or single domain antibodies, are versatile molecules with therapeutic and diagnostic applications, but they are primarily developed by panning nanobody libraries isolated from immunized camelids. This approach is not effective for non-immunogenic or toxic antigens, prevents the use of scaffolds with ideal pharmacokinetic properties, and each target antigen requires its own unique immunization campaign. Accordingly, there is a need to address the aforementioned deficiencies and inadequacies, in particular, relating to targets such as calreticulin (CALR).

### SUMMARY

Described herein are calreticulin-targeting nanobodies, probes comprising calreticulin-targeting nanobodies, compositions comprising such, methods of use, and methods of synthesis.

In examples, a calreticulin-targeting nanobody, comprises a heavy chain variable region comprising (a) a VHCDR1 polypeptide comprising an amino acid sequence having at least 90% sequence identity to SEQ ID NO:14; (b) a VHCDR2 polypeptide comprising an amino acid sequence having at least 90% sequence identity to any one of SEQ ID NOs:4-6 or 10-13; and (c) a VHCDR3 polypeptide comprising an amino acid sequence having at least 90% sequence identity to SEQ ID NO:15.

In examples, a calreticulin-targeting nanobody comprises (a) a VHCDR1 polypeptide comprising an amino acid sequence having at least 95% sequence identity to SEQ ID NO:14; (b) a VHCDR2 polypeptide comprising an amino acid sequence having at least 95% sequence identity to any one of SEQ ID NOs:4-6 or 10-13; and (c) a VHCDR3 polypeptide comprising an amino acid sequence having at least 95% sequence identity to SEQ ID NO:15.

In examples, a calreticulin-targeting nanobody comprises (a) a VHCDR1 polypeptide comprising SEQ ID NO:14; (b) a VHCDR2 polypeptide comprising one of SEQ ID NOs:4-6 or 10-13; and (c) a VHCDR3 polypeptide comprising SEQ ID NO:15.

In examples, a calreticulin-targeting nanobody comprises (a) a VHCDR1 polypeptide comprising SEQ ID NO:14; (b) a VHCDR2 polypeptide comprising SEQ ID NO:6; and (c) a VHCDR3 polypeptide comprising SEQ ID NO:15.

In certain aspects, described herein is a calreticulin-targeting nanobody according to Formula 1 below (N terminus to C terminus):

A-B-X Formula 1

In embodiments, A is one or more paramagnetic ions, one or more x-ray imaging agents, one or more fluorophores, or one or more radioisotopes, individually in or in any combination of any thereof.

In embodiments, B is an optional linker.

In examples, a calreticulin-targeting nanobody of Formula 1 comprises a heavy chain variable region comprising (a) a VHCDR1 polypeptide comprising an amino acid sequence having at least 90% sequence identity to SEQ ID NO:14; (b) a VHCDR2 polypeptide comprising an amino acid sequence having at least 90% sequence identity to any one of SEQ ID NOs:4-6 or 10-13; and (c) a VHCDR3 polypeptide comprising an amino acid sequence having at least 90% sequence identity to SEQ ID NO:15.

In examples, a calreticulin-targeting nanobody of Formula 1 comprises (a) a VHCDR1 polypeptide comprising an amino acid sequence having at least 95% sequence identity to SEQ ID NO:14; (b) a VHCDR2 polypeptide comprising an amino acid sequence having at least 95% sequence identity to any one of SEQ ID NOs:4-6 or 10-13; and (c) a VHCDR3 polypeptide comprising an amino acid sequence having at least 95% sequence identity to SEQ ID NO:15.

In examples, a calreticulin-targeting nanobody of Formula 1 comprises (a) a VHCDR1 polypeptide comprising SEQ ID NO:14; (b) a VHCDR2 polypeptide comprising one of SEQ ID NOs:4-6 or 10-13; and (c) a VHCDR3 polypeptide comprising SEQ ID NO:15.

In examples, a calreticulin-targeting nanobody of Formula 1 comprises (a) a VHCDR1 polypeptide comprising SEQ ID NO:14; (b) a VHCDR2 polypeptide comprising SEQ ID NO:6; and (c) a VHCDR3 polypeptide comprising SEQ ID NO:15.

In embodiments, the one or more fluorophores comprise a near-infrared (NIR) cyanine dye, a hemicyanine dye, a coumarin-based dye, a fluorescein-based dye, a rhodamine-based dye, a BODIPY-Based Dye, a BODIPY-Based NIR Dye, a Xanthene-Based dye, a Xanthene-Based NIR Dye, a cyanine-based dyes, a porphyrin dye, a phthalocyanine dye, or a squaraine dye, individually or in any combination of any thereof.

In embodiments, the one or more fluorophores comprise Alexa 350, Alexa 430, AMCA, BODIPY630/650, BODIPY 650/665, BODIPY-FL, BODPY-R6G, 13BODLPY-TMR, BODLPY-TRX, cascade blue, Cy3, Cy5, 6-FAM, fluorescein isothiocyanate, HEX, 6-JOE, oregon green 488, oregon green 500, oregon green 514, quantum dots, pacific blue, REG, rhodamine green, rhodamine red, renographin, ROX, TAMRA, TET, tetramethylrhodamine, Texas Red, the Alexafluor family, Cy5, Cy5.5, Cy7, indocyanine green (ICG), or one or more fluorescent proteins (e.g., green fluorescent protein (GFP), red fluorescent protein (RFP), and dsRED), individually or in any combination of any thereof.

In embodiments, the radioisotope comprises one or more of ³H, ¹¹C, ¹⁴C, ¹⁸F, ³²P ³⁵S, ³⁶Cl, ⁵¹Cr, ⁵²Fe, ⁵⁷Co, ⁵⁸Co, ⁵⁹Fe, ⁶⁴Cu, ⁶⁷Cu, ⁶⁷Ga, ⁶⁸Ga, ⁷⁵Se, ⁷⁶Br, ⁷⁷Br, Zr⁸⁹, ⁹⁰Y, ^{99m}Tc, ¹¹¹In, ¹²³I, ¹²⁴I, ¹²⁵I, ¹³¹I, ¹⁵²Eu, ¹⁵³Sm, ¹⁶⁶Ho, ¹⁷⁷Lu, ¹⁸⁶Re, ¹⁸⁸Re, ²⁰¹TI, ²⁰³Pb, ²¹⁰At, ²¹¹At, ²¹²Bi, ²¹³Bi, or ²²⁵Ac, individually or in any combination of any thereof.

In embodiments, A further comprises a chelating agent Ch. In embodiments, Ch comprises one or more of 1,4,7-triazacyclononanetriacetic acid (NOTA), 1,4,7,10-tetraazacyclododecane-1,4,7,10-tetraacetic acid (DOTA), 1,4,7-triazacyclononane-1-glutaric acid-4,7-diacetic acid (NODAGA), ethylene diamine tetra-acetic acid (EDTA), diethylene triaminepentaacetic acid (DTPA), cyclohexyl-1,2-diaminetetraacetic acid (CDTA), ethyleneglycol-O,O′-bis(2-aminoethyl)-N,N,N′,N′-tetraacetic acid (EGTA), N,N-bis(hydroxybenzyl)-ethylenediamine-N,N′-diacetic acid (HBED), triethylene tetramine hexaacetic acid (TTHA), hydroxyethyidiamine triacetic acid (HEDTA), 1,4,8,11-tetraazacyclotetradecane-N,N′,N″,N′″-tetraacetic acid (TETA), TMOC, or DFO, individually or in any combination of any thereof. In embodiments, Ch is p-SCN-Bn-NOTA.

In embodiments, B comprises one or more of one or more alkylene groups, one or more amine groups, one or more amide groups, one or more alkyleneoxy groups, one or more thiol groups, one or more carbohydrate groups, or any combination thereof.

In examples, a nanobody comprises C terminus to N terminus formula X-B-A (Formula 2).

In embodiments of Formula 2, A is one or more paramagnetic ions, one or more x-ray imaging agents, one or more fluorophores, or one or more radioisotopes, individually in or in any combination of any thereof.

In embodiments of Formula 2, B is an optional linker.

In examples, a calreticulin-targeting nanobody of Formula 2 comprises a heavy chain variable region comprising (a) a VHCDR1 polypeptide comprising an amino acid sequence having at least 90% sequence identity to SEQ ID NO:14; (b) a VHCDR2 polypeptide comprising an amino acid sequence having at least 90% sequence identity to any one of SEQ ID NOs:4-6 or 10-13; and (c) a VHCDR3 polypeptide comprising an amino acid sequence having at least 90% sequence identity to SEQ ID NO:15.

In examples, a calreticulin-targeting nanobody of Formula 2 comprises (a) a VHCDR1 polypeptide comprising an amino acid sequence having at least 95% sequence identity to SEQ ID NO:14; (b) a VHCDR2 polypeptide comprising an amino acid sequence having at least 95% sequence identity to any one of SEQ ID NOs:4-6 or 10-13; and (c) a VHCDR3 polypeptide comprising an amino acid sequence having at least 95% sequence identity to SEQ ID NO:15.

In examples, a calreticulin-targeting nanobody of Formula 2 comprises (a) a VHCDR1 polypeptide comprising SEQ ID NO:14; (b) a VHCDR2 polypeptide comprising one of SEQ ID NOs:4-6 or 10-13; and (c) a VHCDR3 polypeptide comprising SEQ ID NO:15.

In examples, a calreticulin-targeting nanobody of Formula 2 comprises (a) a VHCDR1 polypeptide comprising SEQ ID NO:14; (b) a VHCDR2 polypeptide comprising SEQ ID NO:6; and (c) a VHCDR3 polypeptide comprising SEQ ID NO:15.

In embodiments of Formula 2, the one or more fluorophores comprise a near-infrared (NIR) cyanine dye, a hemicyanine dye, a coumarin-based dye, a fluorescein-based dye, a rhodamine-based dye, a BODIPY-Based Dye, a BODIPY-Based NIR Dye, a Xanthene-Based dye, a Xanthene-Based NIR Dye, a cyanine-based dyes, a porphyrin dye, a phthalocyanine dye, or a squaraine dye, individually or in any combination of any thereof.

In embodiments of Formula 2, the one or more fluorophores comprise Alexa 350, Alexa 430, AMCA, BODIPY 630/650, BODIPY 650/665, BODIPY-FL, BODPY-R6G, 13BODLPY-TMR, BODLPY-TRX, cascade blue, Cy3, Cy5, 6-FAM, fluorescein isothiocyanate, HEX, 6-JOE, oregon green 488, oregon green 500, oregon green 514, quantum dots, pacific blue, REG, rhodamine green, rhodamine red, renographin, ROX, TAMRA, TET, tetramethylrhodamine, Texas Red, the Alexafluor family, Cy5, Cy5.5, Cy7, indocyanine green (ICG), or one or more fluorescent proteins (e.g., green fluorescent protein (GFP), red fluorescent protein (RFP), and dsRED), individually or in any combination of any thereof.

In embodiments of Formula 2, the radioisotope comprises one or more of ³H, ¹¹C, ¹⁴C, ¹⁸F, ³²P, ³⁵S, ³⁶Cl, ⁵¹Cr, ⁵²Fe, ⁵⁷Co, ⁵⁸Co, ⁵⁹Fe, ⁶⁴Cu, ⁶⁷Cu, ⁶⁷Ga, ⁶⁸Ga, ⁷⁵Se, ⁷⁶Br, ⁷⁷Br, Zr⁸⁹, ⁹⁰Y, ^{99m}Tc, ¹¹¹In, ¹²³I, ¹²⁴I, ¹²⁵I, ¹³¹I, ¹⁵²Eu, ¹⁵3Sm, ¹⁶⁶Ho, ¹⁷⁷Lu, ¹⁸⁶Re, ¹⁸⁸Re, ²⁰¹Tl, ²⁰³Pb, ²¹⁰At, ²¹¹At, ²¹²Bi ²¹³Bi, or ²²⁵Ac, individually or in any combination of any thereof.

In embodiments of Formula 2, A further comprises a chelating agent Ch. In embodiments, Ch comprises one or more of 1,4,7-triazacyclononanetriacetic acid (NOTA), 1,4,7,10-tetraazacyclododecane-1,4,7,10-tetraacetic acid (DOTA), 1,4,7-triazacyclononane-1-glutaric acid-4,7-diacetic acid (NODAGA), ethylene diamine tetra-acetic acid (EDTA), diethylene triaminepentaacetic acid (DTPA), cyclohexyl-1,2-diaminetetraacetic acid (CDTA), ethyleneglycol-O,O′-bis(2-aminoethyl)-N,N,N′,N′-tetraacetic acid (EGTA), N,N-bis(hydroxybenzyl)-ethylenediamine-N,N′-diacetic acid (HBED), triethylene tetramine hexaacetic acid (TTHA), hydroxyethyidiamine triacetic acid (HEDTA), 1,4,8,11-tetraazacyclotetradecane-N,N′,N″,N′″-tetraacetic acid (TETA), TCMC, or DFO, individually or in any combination of any thereof. In embodiments, Ch is p-SCN-Bn-NOTA.

In embodiments of Formula 2, B comprises one or more of one or more alkylene groups, one or more amine groups, one or more amide groups, one or more alkyleneoxy groups, one or more thiol groups, one or more carbohydrate groups, or any combination thereof.

Described herein are methods of using a calreticulin-targeting nanobody.

In embodiments, a method of using a calreticulin-targeting nanobody, comprises: inducing calreticulin (CALR) translocation in a plurality of cells in a biological sample; contacting the sample with a composition as described herein comprising a nanobody described herein or by contacting with the nanobody directly; incubating the contacted sample at a temperature for a period of time; and imaging the incubated sample to determine a nanobody level.

In embodiments, the sample comprises a tumor, a tumor biopsy or suspected tumor in or from a subject. In embodiments, the tumor, tumor biopsy, or suspected tumor comprises an adenocarcinoma, an esophageal cancer, a gastric cancer, a small bowel cancer, a colon cancer, a rectal cancer, an anal cancer, a lunch cancer, a mesothelioma, a peritoneal cancer, an ovarian cancer, a uterine cancer, a breast cancer, a renal cancer, a urothelial cancer, a prostate cancer, a hepatoceullar carcinoma, a cholangiocarcinoma, a lymphoma, a leukemia, a liposarcoma, a, rhabdosarcoma, a fibrosarcoma, an angiosarcoma, a chondrosarcoma, a thyroid cancer, an oral cancer, a head and neck cancer, or a glioblastoma, individually or in any combination of any thereof.

In embodiments, inducing translocation is performed by administering an inducing agent. In embodiments, the inducing agent comprises one or more of an anthracycline, gemcitabine, 5-FU, dantrolene, an oxidizing agent, or a platinium-based inducing agent. In embodiments, the anthracycline comprises doxorubicin. In embodiments, the oxidizing agent comprises hydrogen peroxide.

In embodiments, the imaging is positive emission tomography (PET) scanning.

Described herein are compositions comprising one or more nanobodies as described herein.

In embodiments, a composition described herein comprises one or more calreticulin-targeting nanobodies described herein, and a carrier. In embodiments, the carrier is a pharmaceutically-acceptable carrier.

Described herein are kits comprising one or more nanobodies described herein, and/or a composition as described herein. Kits can further comprise instructions for use. Any one or more aspects of a kit as described herein can be packaged separately from any one or more other components of a kit.

In examples, a nanobody, or nanobody according to Formula 1 or 2 above, can comprise an amino acid sequence having at least 90% to 99%, or 100%, sequence identity to any one of SEQ ID NOs: 20-22.

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIGS. 1A-1F: Generation of Nanobody Library with randomized CDR2 (NaLiC2). FIG. 1A) Schematic of the strategy used to randomize regions of the Nb phage library. FIG. 1B) Workflow of cloning NaLiC2. FIG. 1C) Alignment showing the successful removal of the PfoI restriction site upstream of the nanobody by site directed mutagenesis. FIG. 1D) Agarose gel confirming the Klenow synthesis of the randomized CDR2 insert. FIG. 1E) Agarose gel confirming the complete PfoI and BsgI digestion of the phagemid pGAR2. FIG. 1F) Agarose gel confirming successful ligation of the digested randomized insert and vector.

FIGS. 2A-2D: NaLiC2.21 diversity, selection scheme, and eluted phage. FIG. 2A) Representation of amino acids at each position of CDR2. FIG. 2B) Workflow of library selection for CALR, analysis, and screening. FIG. 2C) Diversity statistics of NaLiC2.21. FIG. 2D) Eluted phage as a percent of added phage from each round of selection.

FIGS. 3A-3D: Analysis of clonal enrichment following NaLiC2.21 selection for CALR. FIG. 3A) Percentage of unique amino acid sequence present in the starting NaLiC2.21 library and after each round of selection. FIG. 3B) The number of reads per million of each CDR2 sequence from the starting library and after each round of selection. Cal1-3 were the top 3 most enriched clones after 3 rounds of selection. The other sequences are the top sequences after round 1 of selection. FIG. 3C) SDS-PAGE of elution 1-3 from Ni2+ agarose purification from Cal1-3. FIG. 3D) Table showing the CDR2 amino acid sequence of Cal1-3 with band intensity of E1 SDS-PAGE in (FIG. 3C). ND: not detectable.

FIGS. 4A-4D: Ca/3 binding to recombinant and cellularcalreticulin. FIG. 4A) ELISA for Cal3 binding to CALR, HER2, and milk. FIG. 4B) Biolayer interferometry analysis of Cal3 binding to hCALR. FIG. 4C) Flow cytometry analysis of Cal3 binding to surface CALR induced by doxorubicin. FIG. 4D) IHC analysis of cellular CALR with the Cal3 Nb and a control Ab for CALR.

FIGS. 5A-5C: In vivo PET imaging of ⁶⁴Cu-NOTA-Ca/3 in mice bearing recombinant hCALR and BSA Matrigelplugs. FIG. 5A) Representative PET/CT images of ⁶⁴Cu-NOTA-Cal3 1-hour post-injection via tail vein. Male C57/B16 (n=4) are bearing hCALR- or BSA-Matrigel plugs on the left and right shoulders, respectively. FIG. 5B) Quantification of PET signal from CALR and BSA plugs. FIG. 5C) Quantification of PET signal from clearance-related organs.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Regarding machine hardware, it should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user/operator/customer/client or machine/system/computer/processor. Moreover, the various components may be in communication via wireless and/or hardwire or other desirable and available communication means, systems and hardware. Moreover, various components and modules may be substituted with other modules or components that provide similar functions.

Embodiments of the present disclosure will employ, unless otherwise indicated, techniques of medicinal chemistry, biochemistry, molecular biology, cellular biology, tissue culture, therapeutic administrations, cancer biology, cancer therapies, cancer diagnostics and the like.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### DEFINITIONS

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject-matter.

The term “about”, when used herein in reference to a value, refers to a value that is similar, in context to the referenced value. In general, those skilled in the art, familiar with the context, will appreciate the relevant degree of variance encompassed by “about” in that context, for example, ±5%, 4%, 3%, 2%, etc.

Two events or entities are “associated” with one another, as that term is used herein, if the presence, level and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

As used herein, the term “comparable” refers to two or more agents, entities, situations, sets of conditions, etc., that may not be identical to one another but that are sufficiently similar to permit comparison there between so that one skilled in the art will appreciate that conclusions can reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Those skilled in the art will appreciate that the term “composition”, as used herein, can be used to refer to a discrete physical entity that comprises one or more specified components. In general, unless otherwise specified, a composition can be of any suitable form—e.g., gel, liquid, solid, etc.

A composition or method described herein as “comprising” one or more named elements or steps is open-ended, meaning that the named elements or steps are essential to a particular aspect or embodiment, but other elements or steps can be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and can also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step can be substituted for that element or step.

In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “Improved,” “increased” or “reduced,” or grammatically comparable comparative terms, indicate values that are relative to a baseline value or reference measurement. For example, in some embodiments, an assessed value achieved with an agent of interest may be “improved” relative to that obtained or expected in the absence of treatment or with a comparable reference agent or control. Alternatively, or additionally, in some embodiments, an assessed value achieved with an agent of interest may be “improved” relative to that obtained in the same subject or system under different conditions (e.g., prior to or after an event such as administration of an agent of interest), or in a different, comparable subject (e.g., in a comparable subject or system that differs from the subject or system of interest). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein, “isolated” means separated from constituents, cellular and otherwise, in which the polynucleotide, peptide, polypeptide, protein, nanobody, antibody, or fragments thereof, are normally associated with in nature. A non-naturally occurring polynucleotide, peptide, polypeptide, protein, nanobody, antibody, or fragments thereof, do not require “isolation” to distinguish it from its naturally occurring counterpart.

As used herein, the term “encode” refers to principle that DNA can be transcribed into RNA, which can then be translated into amino acid sequences that can form proteins

As used herein, the terms “optional” or “optionally” means that the subsequently described event or circumstance can or cannot occur, and that the description includes instances where said event or circumstance occurs and instances where it does not.

As used herein, “individual”, “organism”, “host”, “subject”, and “patient” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). These terms (“individual,” “subject,” “host,” and “patient,” used interchangeably herein also refer to any mammalian subject for whom diagnosis, treatment, or therapy is desired, particularly humans.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

Reference throughout this specification to “one embodiment”, “an embodiment”, “another embodiment”, “some embodiment,” means that a particular feature, structure or characteristic described in connection with the embodiment is included in at least one embodiment of the present invention. Thus, appearances of the phrases “in one embodiment,” “in an embodiment,” “in another embodiment”, or “in some embodiment” in various places throughout this specification are not necessarily all referring to the same embodiment, but they may. Furthermore, the particular features, structures or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the invention. For example, in the appended claims, any of the claimed embodiments can be used in any combination.

A “control” sample or value refers to a sample that serves as a reference, usually a known reference, for comparison to a test sample or condition. For example, a test sample can include cells exposed to a test condition or a test agent, while the control is not exposed to the test condition or agent (e.g., negative control). The control can also be a positive control, e.g., a known primary cell or a cell exposed to known conditions or agents, for the sake of comparison to the test condition. A control can also represent an average value gathered from a plurality of samples, e.g., to obtain an average value. For therapeutic applications, a sample obtained from a patient suspected of having a given disorder or deficiency can be compared to samples from a known normal (non-deficient) individual. A control can also represent an average value gathered from a population of similar individuals, e.g., patient having a given deficiency or healthy individuals with a similar medical background, same age, weight, etc. A control value can also be obtained from the same individual, e.g., from an earlier-obtained sample, prior to the disorder or deficiency, or prior to treatment. One of skill will recognize that controls can be designed for assessment of any number of parameters.

The term “biological sample” encompasses a variety of sample types obtained from an organism or a cell line. The term encompasses blood and other liquid samples of biological origin, solid tissue samples, such as a biopsy specimen or tissue cultures or cells derived therefrom and the progeny thereof. The term includes samples that have been manipulated in any way after their procurement, such as by treatment with reagents, solubilization, or enrichment for certain components. The term includes a clinical sample, and also includes cells in cell culture, cell supernatants, cell lysates, serum, plasma, biological fluids, and tissue samples.

The term “clinical well-being” as used herein, refers to a state or degree of clinical or physiological wellness or health of a patient. A clinician can evaluate a patient's clinical well-being by physical examination or performing one or more tests or assays.

“Inhibitors,” “activators,” and “modulators” of expression or of activity are used to refer to inhibitory, activating, or modulating molecules, respectively, identified using in vitro and in vivo assays for expression or activity of a described target protein (or encoding polynucleotide), e.g., ligands, agonists, antagonists, and their homologs and mimetics. The term “modulator” includes inhibitors and activators. Inhibitors are agents that, e.g., inhibit expression or bind to, partially or totally block stimulation or protease inhibitor activity, decrease, prevent, delay activation, inactivate, desensitize, or down regulate the activity of the described target protein, e.g., antagonists. Activators are agents that, e.g., induce or activate the expression of a described target protein or bind to, stimulate, increase, open, activate, facilitate, enhance activation or protease inhibitor activity, sensitize or up regulate the activity of described target protein (or encoding polynucleotide), e.g., agonists. Modulators include naturally occurring and synthetic ligands, antagonists and agonists (e.g., small chemical molecules, nanobodies and the like that function as either agonists or antagonists). Such assays for inhibitors and activators include, e.g., applying putative modulator compounds to cells expressing the described target protein and then determining the functional effects on the described target protein activity, as described above. Samples or assays comprising described target protein that are treated with a potential activator, inhibitor, or modulator are compared to control samples without the inhibitor, activator, or modulator to examine the extent of effect. Control samples (untreated with modulators) are assigned a relative activity value of 100%. Inhibition of a described target protein is achieved when the activity value relative to the control is about 80%, optionally 50% or 25, 10%, 5% or 1%. Activation of the described target protein is achieved when the activity value relative to the control is 110%, optionally 150%, optionally 200, 300%, 400%, 500%, or 1000-3000% or more higher.

The terms “administering,” “delivering,” and “introducing,” can be used interchangeably to indicate the introduction of a diagnostic or therapeutic composition or agent (e.g., compositions comprising one or more nanobodies described herein) into the body of a subject. The therapeutic composition or agent can be administered through any appropriate means that results in the delivery of at least a portion of the composition or agent to a desired location in the subject such that the composition or agent retains its therapeutic capability. Useful methods of delivering the therapeutic include, but are not limited to, intravenous delivery, subcutaneous delivery, intradermal delivery, intracoronary delivery, intracardiac delivery, oral delivery, or any combination thereof.

The term “administered continuously” refers to the continuous delivery of a therapeutic agent, e.g., compound, molecule, peptide, biologic, chemical, etc. over a 24-hour period.

The term “therapeutically effective amount” refers to an amount of therapeutic agent effective to treat at least one symptom of a disease or disorder in a subject. In other words, such an amount is sufficient to bring about a beneficial or desired clinical effect. The “therapeutically effective amount” of the agent for administration may vary based upon the desired activity, the diseased state of the subject being treated, the dosage form, method of administration, subject factors such as the subject's sex, genotype, weight and age, the underlying causes of the condition or disease to be treated, the route of administration and bioavailability, the persistence of the administered agent in the body, evidence of natriuresis and/or diuresis, the type of formulation, and the potency of the agent.

As used herein, the terms “pharmaceutically acceptable” or “pharmacologically acceptable” refer to compositions that do not substantially produce adverse reactions, e.g., toxic, allergic, or immunological reactions, when administered to a subject.

The terms “therapy,” “treatment,” and “amelioration” refer to any reduction in the severity of symptoms, e.g., of a neurodegenerative disorder or neuronal injury. As used herein, the terms “treat” and “prevent” are not intended to be absolute terms. Treatment can refer to any delay in onset, amelioration of symptoms, improvement in patient survival, improved cognitive function or coordination, increase in survival time or rate, etc. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

As used throughout, the terms “nucleic acid,” “nucleic acid sequence,” “oligonucleotide,” “nucleotides,” or other grammatical equivalents as used herein mean at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, cRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

Unless otherwise indicated, a particular nucleic acid sequence also implicitly encompasses conservatively modified variants thereof, alleles, orthologs, SNPs, and complementary sequences as well as the sequence explicitly indicated.

As used herein, “cDNA” refers to a DNA sequence that is complementary to an RNA transcript in a cell. It is a man-made molecule. Typically, cDNA is made in vitro by an enzyme called reverse-transcriptase using RNA transcripts as templates.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88).

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L- and D-amino acids. The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions. The terms “portion” and “fragment” are used interchangeably herein to refer to parts of a polypeptide, nucleic acid, or other molecular construct.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23(4): 581-87 (2013); Xie et al. “Adding amino acids to the genetic repertoire,” Curr. Opin. Chem. Biol. 9(6): 548-54 (2005); and all references cited therein. Beta and gamma amino acids are known in the art and are also contemplated herein as unnatural amino acids.

In accordance with standard nomenclature, amino acid residue sequences are denominated by either a three letter or a single letter code as indicated as follows, for example: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be involved in the structure, function, and regulation of various functions.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith & Waterman Add. APL. Math. 2:482 (1981), by the homology alignment algorithm of Needleman & Wunsch J. Mol. Biol. 48:443 (1970), by the search for similarity method of Pearson & Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-10 and Altschul et al. (1977) Nucleic Acids Res. 25: 3389-402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1977)). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

As used herein, the term “antibody” encompasses antibodies and antibody fragments thereof, derived from any antibody-producing mammal (e.g., mouse, rat, rabbit, and primate including human), or from a hybridoma, phage selection, recombinant expression or transgenic animals (or other methods of producing antibodies or antibody fragments”), that specifically bind to a target polypeptide, such as, for example, CALR, polypeptides or portions thereof. It is not intended that the term “antibody” limited as regards to the source of the antibody or the manner in which it is made (e.g., by hybridoma, phage selection, recombinant expression, transgenic animal, peptide synthesis, etc.). Exemplary antibodies include polyclonal, monoclonal and recombinant antibodies; pan-specific, multispecific antibodies (e.g., bispecific antibodies, trispecific antibodies); humanized antibodies; murine antibodies; chimeric, mouse-human, mouse-primate, primate-human monoclonal antibodies; and anti-idiotype antibodies and may be any intact antibody or fragment thereof. As used herein, the term “antibody” encompasses not only intact polyclonal or monoclonal antibodies, but also fragments thereof (such as dAb, Fab, Fab′, F(ab′) 2, Fv), single chain (ScFv), synthetic variants thereof, naturally occurring variants, fusion proteins comprising an antibody portion with an antigen-binding fragment of the required specificity, humanized antibodies, chimeric antibodies, and any other modified configuration of the immunoglobulin molecule that comprises an antigen-binding site or fragment (epitope recognition site) of the required specificity. A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific for the target antigen. The term “monoclonal antibody” encompasses not only intact monoclonal antibodies and full-length monoclonal antibodies, but also fragments thereof (such as Fab, Fab′, F(ab′) 2, Fv), single chain (ScFv), variants thereof, fusion proteins comprising an antigen-binding portion, humanized monoclonal antibodies, chimeric monoclonal antibodies, and any other modified configuration of the immunoglobulin molecule that comprises an antigen binding fragment (epitope recognition site) of the required specificity and the ability to bind to an epitope. It is not intended to be limited as regards the source of the antibody or the manner in which it is made (e.g., by hybridoma, phage selection, recombinant expression, transgenic animals, etc.). The term includes whole immunoglobulins as well as the fragments etc. described above under the definition of “antibody”. As used herein, the term “antibody fragment” refers to a portion derived from or related to a full-length antibody, such as, for example, an anti-CALR antibody, generally including the antigen binding or variable region thereof. Illustrative examples of antibody fragments include Fab, Fab′, F(ab) 2, F(ab′) 2 and Fv fragments, scFv fragments, diabodies, linear antibodies, single-chain antibody molecules and multispecific antibodies formed from antibody fragments. As used herein, a “single-chain Fv” or “scFv” antibody fragment comprises the VH and VL domains of an antibody, wherein these domains are present in a single polypeptide chain. Generally, the Fv polypeptide further comprises a polypeptide linker between the VH and VL domains, which enables the scFv to form the desired structure for antigen binding. As used herein, a “chimeric antibody” is a recombinant protein that contains the variable domains and complementarity-determining regions derived from a non-human species (e.g., rodent) antibody, while the remainder of the antibody molecule is derived from a human antibody.

The term “nanobody,” also known as a single-domain antibody (sdAb) or VHH, is an antibody fragment consisting of a single monomeric variable antibody domain derived from the heavy chain of camelids.

### Discussion

Described herein are novel calreticulin (CALR)-targeting probes, methods of use, and kits comprising such.

Such aspects of the present disclosure can provide novel imaging tools and methodologies utilize such that are capable, for example, of tracing cells in which CALR has (or can be) translocated to the membrane. Nanobodies as described herein can be utilized, for example, as a diagnostic for non-invasive imaging PET, but for surgical applications, fluorophores can be utilized in place of radioisotopes.

### I. CALRETICULIN-TARGETED NANOBODIES

Described herein are novel calreticulin (CALR)-targeting (also referred to herein as CALR-binding) nanobodies and antigen-binding fragments thereof. Such nanobodies can be useful, for example, in diagnostic applications for cancers. In examples, nanobody as used herein also refers to an antigen-binding fragment of a nanobody described herein.

Nanobodies, derived from the variable domain of heavy-chain-only antibodies found in camelids, are compact and versatile antibody fragments that have emerged as powerful tools with significant implications in therapy and research. Their unique structural features, characterized by a single monomeric domain comprised of a rigid scaffold and defined binding regions, endow nanobodies with high stability, solubility, and binding specificity to a diverse array of targets. In imaging applications, nanobodies exhibit advantages such as improved tissue penetration and rapid clearance compared to traditional antibody therapies, making them attractive candidates for non-invasive diagnostic agents.

Nanobodies are typically discovered through immunization to a target antigen of interest. Generated nanobody sequences can be cloned into phage display libraries that allow for the panning of nanobodies that bind the target of interest. While immunized phage display libraries have proven to be a valuable method for nanobody discovery, they are not without their limitations. Non-immunogenic antigens, molecules with structures that are not stable or unable to be formulated for immunization, and inability to direct selectivity towards specific epitopes all represent challenges to existing immunization strategies. Synthetic phage display libraries offer several advantages that directly address these limitations. Additionally, the controlled and systematic design of synthetic libraries allows for the incorporation of specific structural features, such as varied complementarity-determining region (CDR) lengths and amino acid compositions, enhancing the range of desired nanobody properties. This rational engineering enables the selection of nanobodies against targets that may be challenging or impractical to use in immunization strategies, including toxic or non-immunogenic antigens. Moreover, synthetic libraries are not limited by species-specific immune responses, making them versatile tools for targeting a diverse array of antigens.

Described herein are calreticulin-targeting nanobodies and compositions, methods, and kits thereof.

In embodiments, a composition comprising a calreticulin-targeting nanobody according to Formula 1 below (N terminus to C terminus):

A-B-X Formula 1

wherein A is a fluorochrome, caged fluorochrome, or radioisotope; wherein B is an optional linker; and wherein X comprises an anti-calreticulin nanobody comprising a CDR1, CDR2, and CDR3 sequence. Formula 1 above is shown as the non-limiting example of N terminus to C terminus. The skilled artisan would understand that other arrangements of elements A, B, and X are possible, for example and without intending to be limiting, a C terminus to N terminus formula X-B-A (Formula 2).

### A. Calreticulin-Targeting Nanobodies

In embodiments, an anti-calreticulin nanobody or antigen-binding fragment thereof can comprise a heavy chain CDR1, CDR2, and CDR3 sequence and other aspects as described below.

In embodiments, an anti-calreticulin nanobody or antigen-binding fragment thereof can target Calreticulin. In embodiments, the Calreticulin is human Calreticulin (NCBI Gene ID:811 and NCBI Reference Sequence: NP_004334.1, both gene and protein sequences of which are incorporated by reference as if fully set forth herein). In embodiments, an anti-calreticulin nanobody or antigen-binding fragment thereof can target the extracellular component of a human Calreticulin protein.

i. Calreticulin-Targeting Nanobodies and Compositions Thereof

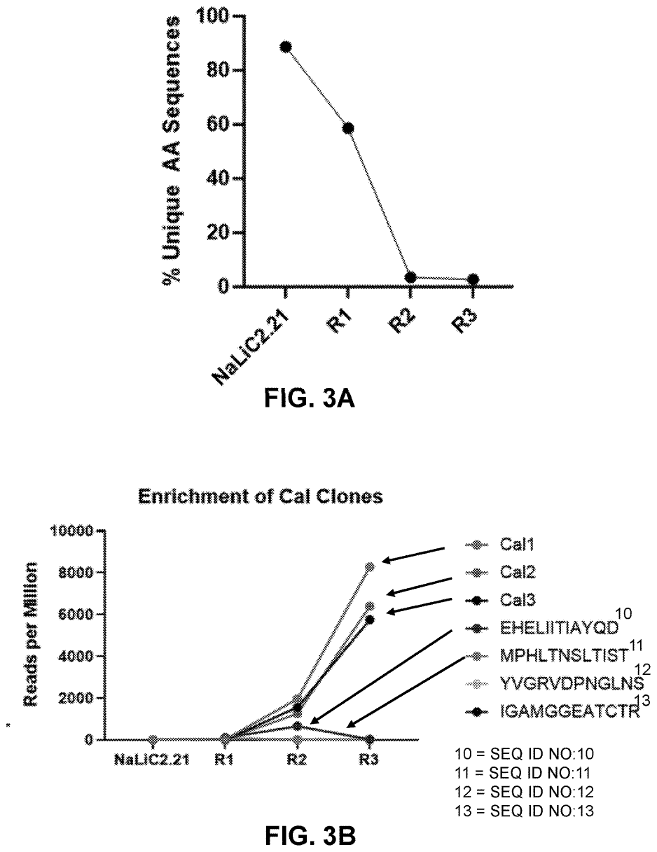

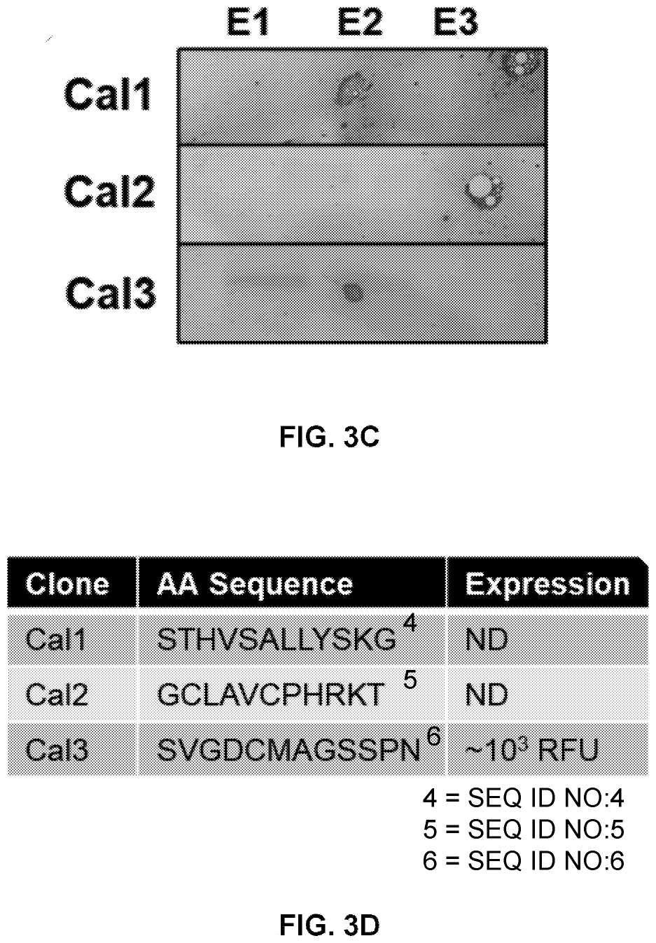

Nanobodies that specifically or selectively binds CALR are provided herein, as well as compositions thereof (i.e., a composition comprising, for example, an aqueous carrier or vehicle such as water or phosphate-buffered saline (PBS)). In certain aspects, nanobodies as described herein are human or rodent antibody fragments derived from a camelid. In embodiments of the present disclosure, novel nanobodies specifically bind to CALR as a tumor antigen. Embodiments of such comprise those shown in FIGS. 3A-3D and in the informal sequence listing below.

As used herein, the terms specifically binds to, specific for, selectively binds and selective for CALR or a CALR antigen means binding that is measurably different from a non-specific or non-selective interaction. Specific binding can be measured, for example, by determining binding of a molecule compared to binding of a control molecule. Specific binding can also be determined by competition with a control molecule that is similar to the target, such as an excess of non-labeled target. In that case, specific binding is indicated if the binding of the labeled target to a probe is competitively inhibited by the excess non-labeled target. In that case, specific binding is indicated if the binding of the labeled target to a probe is competitively inhibited by the excess non-labeled target. The term “specifically binds”, as used herein, when referring to a polypeptide (including nanobodies) or receptor, refers to a binding reaction which is determinative of the presence of the protein or polypeptide or receptor in a heterogeneous population of proteins and other biologics. Thus, under designated conditions (e.g., immunoassay conditions in the case of an nanobody), a specified ligand or nanobody “specifically binds” to its particular “target” (e.g. a nanobody specifically binds to an endothelial antigen) when it does not bind in a significant amount to other proteins present in the sample or to other proteins to which the ligand or nanobody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

A nanobody, as used herein, can refer to an antigen binding fragment derived from the variable region of the heavy chain of a heavy-chain only antibody. Antigen binding fragments can comprise at least one antigen binding domain. One example of an antigen binding domain is an antigen binding domain formed by any one or more of CDR1, CDR2, or CDR3. Nanobodies and antigen binding fragments can be described by the antigen to which they specifically bind.

Nanobodies can have one or more complementarity determining regions (CDRs) interspersed with regions that are more conserved. The more conserved regions are called framework regions (FRs). Each nanobody generally comprises three CDRs and four FRs, arranged in the following order (from N-terminus to C-terminus): FR1-CDR1-FR2-CDR2-FR3-CDR3-FR4. The CDRs are involved in antigen binding and confer antigen specificity and binding affinity to the nanobody. (See Kabat et al. (1991) Sequences of Proteins of Immunological Interest 5th ed., Public Health Service, National Institutes of Health, Bethesda, MD.).

The V_{H} region of the nanobodies can be further subdivided into regions of hypervariability (hypervariable regions (HVRs), also called complementarity determining regions (CDRs)) interspersed with regions that are more conserved. The more conserved regions are called framework regions (FRs). Each V_{H} generally comprises three CDRs and four FRs, arranged in the following order (from N-terminus to C-terminus): FR1-CDR1-FR2-CDR2-FR3-CDR3-FR4. The CDRs are involved in antigen binding and confer antigen specificity and binding affinity to the nanobody. (See Kabat et al. (1991) Sequences of Proteins of Immunological Interest 5th ed., Public Health Service, National Institutes of Health, Bethesda, MD.) CDR sequences on the heavy chain (V_{H}) nanobody may be designated as CDRH1, 2, 3.

The term epitope, as used herein, means a component of an antigen (such as a CALR or catalytically-active fragment thereof) capable of specific binding to a nanobody or antigen binding fragment thereof. Such components optionally comprise one or more contiguous amino acid residues and/or one or more non-contiguous amino acid residues. Epitopes frequently consist of surface-accessible amino acid residues and/or sugar side chains and can have specific three-dimensional structural characteristics, as well as specific charge characteristics. Conformational and non-conformational epitopes are distinguished in that the binding to the former but not the latter is lost in the presence of denaturing solvents. An epitope can comprise amino acid residues that are directly involved in the binding, and other amino acid residues, which are not directly involved in the binding. The epitope to which an antigen binding protein binds can be determined using known techniques for epitope determination such as, for example, testing for antigen binding protein binding to antigen variants with different point mutations.

Provided herein are nanobodies or antigen binding portions thereof that specifically bind to antigens related to CALR. Such nanobodies, or VHH (Variable domain of Heavy-chain antibody) molecules, may be, for example, single-domain nanobody fragments derived from the heavy-chain-only antibodies (HCAbs) found in camelids or recombinant/chimeric nanobodies (i.e., synthetic nanobodies derived from synthetic nucleic acid constructs, such as viral vectors, that may also contain monoclonal nanobody sequences as disclosed herein). In particular, antigens can be CALR or portions thereof and regions of CALR that may extend from the extracellular surface of the cellular membrane, be interspersed within the cellular membrane, or extend from the cellular membrane to the intracellular space.

In each case, where a specific amino acid sequence is recited, embodiments comprising a CDR2 with a sequence having at least 90% (e.g. 90%, 91%, 92%, 93%, 94%, 95%, 96%, 97%, 98%, or 99%) identity to the recited sequence (e.g., SEQ ID NOs: 4-6 or 10-13) are also provided.

In each case, where a specific amino acid sequence is recited, embodiments consisting essentially of a CDR2 with a sequence having at least 90% (e.g. 90%, 91%, 92%, 93%, 94%, 95%, 96%, 97%, 98%, or 99%) identity to the recited sequence (e.g., SEQ ID NOs: 4-6 or 10-13) are also provided.

In each case, where a specific amino acid sequence is recited, embodiments consisting of a CDR2 with a sequence having at least 90% (e.g. 90%, 91%, 92%, 93%, 94%, 95%, 96%, 97%, 98%, or 99%) identity to the recited sequence (e.g., SEQ ID NOs: 4-6 or 10-13) are also provided.

In each case, where a specific CDR2 is recited, embodiments comprising, consisting essentially of, or consisting of a CDR2 with a sequence of any of SEQ ID NOs: 4-6 or 10-13.

In each case, where a specific amino acid sequence is recited, embodiments comprising a CDR1 with a sequence having at least 90% (e.g. 90%, 91%, 92%, 93%, 94%, 95%, 96%, 97%, 98%, or 99%) identity to the recited sequence (e.g., SEQ ID NO:14) are also provided.

In each case, where a specific amino acid sequence is recited, embodiments consisting essentially of a CDR1 with a sequence having at least 90% (e.g. 90%, 91%, 92%, 93%, 94%, 95%, 96%, 97%, 98%, or 99%) identity to the recited sequence (e.g., SEQ ID NO:14) are also provided.

In each case, where a specific amino acid sequence is recited, embodiments consisting of a CDR1 with a sequence having at least 90% (e.g. 90%, 91%, 92%, 93%, 94%, 95%, 96%, 97%, 98%, or 99%) identity to the recited sequence (e.g., SEQ ID NO:14) are also provided.

In each case, where a specific CDR1 is recited, embodiments can comprise, consist essentially of, or consist of a CDR3 of SEQ ID NO:14.

In each case, where a specific amino acid sequence is recited, embodiments comprising a CDR3 with a sequence having at least 90% (e.g. 90%, 91%, 92%, 93%, 94%, 95%, 96%, 97%, 98%, or 99%) identity to the recited sequence (e.g., SEQ ID NO:15) are also provided.

In each case, where a specific amino acid sequence is recited, embodiments consisting essentially of a CDR3 with a sequence having at least 90% (e.g. 90%, 91%, 92%, 93%, 94%, 95%, 96%, 97%, 98%, or 99%) identity to the recited sequence (e.g., SEQ ID NO:15) are also provided.

In each case, where a specific amino acid sequence is recited, embodiments consisting of a CDR3 with a sequence having at least 90% (e.g. 90%, 91%, 92%, 93%, 94%, 95%, 96%, 97%, 98%, or 99%) identity to the recited sequence (e.g., SEQ ID NO:15) are also provided.

In each case, where a specific CDR3 is recited, embodiments can comprise, consist essentially of, or consist of a CDR3 of SEQ ID NO:15.

The skilled artisan would recognize that a nanobody having a CDR1, CDR2, and CDR3 sequence can have any combination of CDR1, CDR2, and CDR3 described individually above.

In each case, where a specific amino acid sequence is recited, embodiments comprising a CALR-targeting nanobody with a sequence having at least 90% (e.g. 90%, 91%, 92%, 93%, 94%, 95%, 96%, 97%, 98%, or 99%) identity to the recited sequence (e.g., one of SEQ ID NOs:20-22) are also provided.

In each case, where a specific amino acid sequence is recited, embodiments consisting essentially of of a CALR-targeting nanobody with a sequence having at least 90% (e.g. 90%, 91%, 92%, 93%, 94%, 95%, 96%, 97%, 98%, or 99%) identity to the recited sequence (e.g., one of SEQ ID NOs:20-22) are also provided.

In each case, where a specific amino acid sequence is recited, embodiments consisting of a CALR-targeting nanobody with a sequence having at least 90% (e.g. 90%, 91%, 92%, 93%, 94%, 95%, 96%, 97%, 98%, or 99%) identity to the recited sequence (e.g., one of SEQ ID NOs:20-22) are also provided.

As described herein, a nanobody can comprise, consist essentially of, or consist of a full length sequence of any one of SEQ ID NOs: 20-22.

The amino acid residue sequences provided herein are set forth in single-letter amino acid code which can be used interchangeably with three-letter amino acid code. An amino acid refers to any monomer unit that can be incorporated into a peptide, polypeptide, or protein. The twenty natural or genetically encoded alpha-amino acids are as follows: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (lie or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). The structures of these twenty natural amino acids are shown in, e.g., Stryer et al., Biochemistry, 5^{th} ed., Freeman and Company (2002). The term amino acid also includes unnatural amino acids, modified amino acids (e.g., having modified side chains and/or backbones), and amino acid analogs.

As with all peptides, polypeptides, and proteins, including fragments thereof, it is understood that additional modifications in the amino acid sequence of the CALR antigen-specific nanobodies or antigen binding fragments thereof described herein, for example, in the heavy chain variable region can occur that do not alter the nature or function of the nanobodies or antigen binding fragments thereof. Such modifications include conservative amino acids substitutions, such that each recited sequence optionally contains one or more conservative amino acid substitutions. The list provided below identifies examples of groups that contain amino acids that are conservative substitutions for one another; these groups are exemplary as other conservative substitutions are known to those of skill in the art.
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (WN);
7) Serine (S), Threonine (T); and
8) Cysteine (C), Methionine (M)

By way of example, when an aspartic acid at a specific residue is mentioned, also contemplated is a conservative substitution at the residue, for example, glutamic acid. Non-conservative substitutions, for example, substituting a proline with glycine, are also contemplated.

In some instances, the affinity of a CALR nanobody thereof may be optimized through mutations to increase or decrease affinity as desired based on one or more of the known characteristics of the binding interaction with the cognant CALR antigen. In some instances, the mutations permit facile elution of purified nanobodies or fragments thereof under desirable elution conditions during isolation and purification.

Methods of generating and screening for nanobodies and antigen binding fragments thereof as provided in this disclosure are described in the Examples and are well-known in the art.

The present disclosure also encompasses nanobody fragments that bind to the same epitope of CALR antigens as the nanobodies disclosed herein. Such nanobodies can be identified using routine techniques known in the art, including, for example, competitive binding assays.

A human nanobody is one that possesses an amino acid sequence corresponding to that of an antibody heavy chain or nanobody produced by a human or a human cell, or derived from a non-human source that utilizes a human antibody repertoire or human antibody-encoding sequences (e.g., obtained from human sources, genetically modified non-human sources or designed de novo). Human antibodies specifically exclude humanized antibodies. In certain aspects, the nanobodies of the present disclosure may be human or rodent, or otherwise humanized or otherwise optimized to bind in both humans and rodents (mice in particular).

Nanobodies can be from any class of antibodies including, but not limited to, IgG, IgA, IgM, IgD, and IgE, and from any subclass (e.g., IgG1, IgG2, IgG3, and IgG4) of antibodies. The preparation of nanobody molecules can be monoclonal or polyclonal. A nanobody can also be a human, humanized, CDR-grafted, or an in vitro generated nanobody fragment. The nanobody can have a heavy chain constant region chosen from, e.g., IgG1, IgG2, IgG3, or IgG4.

In some embodiments, the nanobody is monoclonal. As used herein, the term monoclonal nanobody refers to a nanobody from a population of substantially homogeneous nanobodies. A population of substantially homogeneous antibodies comprises antibodies that are the same or substantially similar and that bind the same epitope(s), except for variants that can normally arise during production of the monoclonal nanobody. Such variants are generally present in only minor amounts. A monoclonal nanobody is typically obtained by a process that includes the selection of a single nanobody from a plurality of antibodies. For example, the selection process can be the selection of a unique clone from a plurality of clones, such as a pool of yeast clones, phage clones, bacterial clones, mammalian cell clones, hybridoma clones, or other recombinant DNA clones. The selected nanobody can be further altered, for example, to improve affinity for the target, for example, by affinity maturation, to humanize the nanobody, to improve its production in cell culture, and/or to reduce its immunogenicity in a subject.

Nanobodies can be single domain antibody fragments. Single domain nanobodies can include nanobodies whose complementary determining regions are part of a single domain polypeptide. Examples include, but are not limited to, heavy chain nanobodies, nanobodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, rat, guinea, pig, human, camel, llama, fish, shark, goat, rabbit, and bovine. Single domain antibodies are described, for example, in International Application Publication No. WO 94/04678. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species (e.g., camel, llama, dromedary, alpaca and guanaco) or other species besides Camelidae.

In certain embodiments, nanobodies and nanobody compositions as provided herein are distinguishable from naturally occurring antibodies and compositions in one or more respects. Such distinguishable antibodies and compositions may be referred to as “synthetic,” or may be identified by the proviso that the nanobody or composition “is not naturally occurring” or affirmatively as “non-naturally occurring.” Possible differences for synthetic antibodies include variable region sequences that differ corresponding naturally occurring antibodies, different isotypes, different allotypes, and different constant domain variants. These differences are discussed in more detail below. In some embodiments, the synthetic nanobody is an engineered polypeptide, also referred to as a recombinant polypeptide, that is made using conventional protein and nanobody engineering molecular biology, chemical, and biochemical methods as described below, including, but not limited to, those described in the Examples below.

In some embodiments, the nanobody comprises a heavy chain variable region sequence that is derived from an immunoglobulin-producing human B cell, and further comprises a heavy chain constant region having an IgG isotype (e.g., IgG4), an IgA isotype (e.g., IgA1), an IgM isotype, an IgD isotype, or that is derived from an IgG, IgA, IgM, or IgD isotype (e.g., is a modified IgG4 constant region). It will be appreciated by a person of ordinary skill in the art that the different heavy chain isotypes (IgA, IgD, IgE, IgG, and IgM) have different effector functions that are mediated by the heavy chain constant region, and that for certain uses it may be desirable to have a nanobody that has the effector function of a particular isotype (e.g., IgG).

Synthetic nanobodies and synthetic antigen-binding fragments of this disclosure may comprise variations in heavy chain constant regions to change the properties of the synthetic nanobody relative to the corresponding naturally occurring nanobody. Exemplary changes include mutations to modulate nanobody effector function (e.g., complement-based effector function or FcγR-based effector function), alter half-like, modulate co-engagement of antigen and FcγRs, introduce or remove glycosylation motifs (glyco-engineering). See Fonseca et al., 2018, “Boosting half-life and effector functions of therapeutic antibodies by Fc-engineering: An interaction-function review” Int J Biol Macromol. 19:306-311; Wang et al., 2018, “IgG Fc engineering to modulate antibody effector functions” Protein Cell 2018, 9(1):63-73; Schlothauer, 2016, “Novel human IgG1 and IgG4 Fc-engineered antibodies with completely abolished immune effector functions,” Protein Engineering, Design and Selection 29(10):457-466; Tam et al., 2017, “Functional, Biophysical, and Structural Characterization of Human IgG1 and IgG4 Fc Variants with Ablated Immune Functionality” Antibodies 6, 12, each incorporated herein by reference for all purposes.

The heavy chain variable region sequence of nanobody described herein can be engineered to comprise one or more variations in the heavy chain variable region sequence. In some embodiments, the engineered variation(s) improves the binding affinity of the nanobody for a CALR. In some embodiments, the engineered variation(s) improves the cross-reactivity of the nanobody for a CALR antigen.

In some embodiments, the engineered variation is a variation in one or more CDRs, e.g., an amino acid substitution in a heavy chain CDR as described herein. In some embodiments, the engineered variation is a variation in one or more framework regions, e.g., an amino acid substitution in a heavy chain framework region. In some embodiments, the engineered variation is a reversion of a region of the heavy chain sequence to the inferred naïve sequence. Methods for determining an inferred naïve immunoglobulin sequence are described in the art. See, e.g., Magnani et al., PLoS Negl Trop Dis, 2017, 11:e0005655, doi:10.1371/journal.pntd.0005655

In some embodiments, affinity maturation is used to engineer further mutations that enhance the binding affinity of the nanobody for a CALR antigen or enhance the cross-reactivity of the nanobody for a second CALR antigen. Methods for performing affinity maturation are known in the art. See, e.g., Renaut et al., Methods Mol Biol, 2012, 907:451-461.

In some embodiments, an antigen binding fragment can also be or can also comprise, e.g., a non-antibody, scaffold protein. These proteins are generally obtained through combinatorial chemistry-based adaptation of preexisting antigen-binding proteins. For example, the binding site of human transferrin for human transferrin receptor can be diversified using the system described herein to create a diverse library of transferrin variants, some of which have acquired affinity for different antigens. See, e.g., Ali et al. (1999) J. Biol. Chem. 274:24066-24073. The portion of human transferrin not involved with binding the receptor remains unchanged and serves as a scaffold, like framework regions of nanobodies, to present the variant binding sites. The libraries are then screened, as an nanobody library is screened, and in accordance with the methods described herein, against a target antigen of interest to identify those variants having optimal selectivity and affinity for the target antigen. See, e.g., Hey et al. (2005) TRENDS Biotechnol 23(10):514-522.

Synthetic nanobodies of this disclosure may differ from naturally occurring compositions in at least one or more of the following respects: (i) composition comprises nanobodies that are purified, i.e., separated from tissue or cellular material with which they are associated in the human body, and optionally in an manufactured excipient or medium; and/or (ii) nanobody compositions according to the present disclosure contain a single species of nanobody (are monoclonal) such that all nanobodies in the composition have the same structure and specificity.

ii. Expression and Purification of Nanobodies

The CALR-binding nanobodies (and antigen-binding fragments thereof) disclosed herein may be produced by recombinant expression in a human or non-human cell. Synthetic nanobody-producing cells include non-human cells expressing heavy chains; human cells that are not immune cells; heavy chains; and human B cells that produce heavy chains. Synthetic nanobodies of this disclosure may be heterologously expressed, in vitro or in vivo, in cells other than human B cells, such as non-human cells and human cells other than B cells, optionally other than immune cells, and optionally in cells other than cells in a B cell lineage.

The CALR-binding nanobodies (and antigen-binding fragments thereof) disclosed herein can be produced using a variety of techniques known in the art of molecular biology and protein chemistry. For example, a nucleic acid encoding the nanobody can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system, such that it can be maintained in two different organisms, for example, in mammalian or insect cells for expression and in a prokaryotic host for cloning and amplification.

Several possible vector systems are available for the expression of cloned nanobody polypeptides from nucleic acids in mammalian cells. One class of vectors relies upon the integration of the desired gene sequences into the host cell genome. Cells that have stably integrated DNA can be selected by simultaneously introducing drug resistance genes such as E. coli gpt (Mulligan and Berg (1981) Proc Natl Acad Sci USA 78:2072) or Tn5 neo (Southern and Berg (1982) Mol Appl Genet 1:327). The selectable marker gene can be either linked to the DNA gene sequences to be expressed or introduced into the same cell by co-transfection (Wigler et al. (1979) Cell 16:77). A second class of vectors utilizes DNA elements that confer autonomously replicating capabilities to an extrachromosomal plasmid. These vectors can be derived from animal viruses, such as bovine papillomavirus (Sarver et al. (1982) Proc Natl Acad Sci USA, 79:7147), CMV, polyoma virus (Deans et al. (1984) Proc Natl Acad Sci USA 81:1292), or SV40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO₄ precipitation, liposome fusion, cationic liposomes, electroporation, nucleoporation, viral infection, dextran-mediated transfection, polybrene-mediated transfection, protoplast fusion, and direct microinjection.

Appropriate host cells for the expression of nanobodies include yeast, bacteria, insect, plant, and mammalian cells. Of particular interest are bacteria such as E. coli, fungi such as Saccharomyces cerevisiae and Pichia pastoris, insect cells such as SF9, mammalian cell lines (e.g., human cell lines), as well as primary cell lines.

In some embodiments, a nanobody can be expressed in, and purified from, transgenic animals (e.g., transgenic mammals). For example, a nanobody can be produced in transgenic non-human mammals (e.g., rodents) and isolated from milk as described in, e.g., Houdebine (2002) Curr Opin Biotechnol 13(6):625-629; van Kuik-Romeijn et al. (2000) Transgenic Res 9(2):155-159; and Pollock et al. (1999) J Immunol Methods 231(1-2):147-157.

The nanobodies can be produced from the cells by culturing a host cell transformed with the expression vector containing nucleic acid encoding the nanobodies, under conditions, and for an amount of time, sufficient to allow expression of the proteins. Such conditions for protein expression vary with the choice of the expression vector and the host cell and are easily ascertained by one skilled in the art through routine experimentation. For example, nanobodies expressed in E. coli can be refolded from inclusion bodies (see, e.g., Hou et al. (1998) Cytokine 10:319-30). Bacterial expression systems and methods for their use are known in the art (see Ausubel et al. (1988) Current Protocols in Molecular Biology, Wiley & Sons; and Green and Sambrook (2012) Molecular Cloning—A Laboratory Manual, 4th Ed., Cold Spring Harbor Laboratory Press, New York (2001)). The choice of codons, suitable expression vectors and suitable host cells vary depending on a number of factors and may be easily optimized as needed. A nanobody described herein can be expressed in mammalian cells or in other expression systems including but not limited to yeast, baculovirus, and in vitro expression systems (see, e.g., Kaszubska et al. (2000) Protein Expression and Purification 18:213-220).

One method of producing proteins comprising the provided nanobodies is to link two or more peptides or polypeptides together by protein chemistry techniques (or recombinant DNA techniques). For example, peptides or polypeptides can be chemically synthesized using currently available laboratory equipment using either Fmoc (9-fluorenylmethyl-oxycarbonyl) or Boc (tert-butyloxycarbonoyl) chemistry (Applied Biosystems, Inc.; Foster City, CA). Those of skill in the art readily appreciate that a peptide or polypeptide corresponding to the nanobody provided herein, for example, can be synthesized by standard chemical reactions. For example, a peptide or polypeptide can be synthesized and not cleaved from its synthesis resin whereas the other fragment of a nanobody can be synthesized and subsequently cleaved from the resin, thereby exposing a terminal group that is functionally blocked on the other fragment. By peptide condensation reactions, these two fragments can be covalently joined via a peptide bond at their carboxyl and amino termini, respectively, to form a nanobody or fragment thereof. (Grant GA (1992) Synthetic Peptides: A User Guide. W.H. Freeman and Co., N.Y. (1992); Bodansky M and Trost B., Ed. (1993) Principles of Peptide Synthesis. Springer Verlag Inc., NY). Alternatively, the peptide or polypeptide can by independently synthesized in vivo. Once isolated, these independent peptides or polypeptides may be linked to form an nanobody or fragment thereof via similar peptide condensation reactions.

For example, enzymatic ligation of cloned or synthetic peptide segments can allow relatively short peptide fragments to be joined to produce larger peptide fragments, polypeptides, or whole protein domains (Abrahmsen et al., Biochemistry, 30:4151 (1991)). Alternatively, native chemical ligation of synthetic peptides can be utilized to synthetically construct large peptides or polypeptides from shorter peptide fragments. This method consists of a two-step chemical reaction (Dawson et al., Science, 266:776 779 (1994)). The first step is the chemoselective reaction of an unprotected synthetic peptide a thioester with another unprotected peptide segment containing an amino terminal Cys residue to give a thioester linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini et al., FEBS Lett. 307:97-101 (1992); Clark et al., J. Biol. Chem. 269:16075 (1994); Clark et al., Biochemistry 30:3128 (1991); Rajarathnam et al., Biochemistry 33:6623-30 (1994)).

Alternatively, unprotected peptide segments can be chemically linked where the bond formed between the peptide segments as a result of the chemical ligation is an unnatural (non-peptide) bond (Schnolzer et al., Science 256:221 (1992)). This technique has been used to synthesize analogs of protein domains as well as large amounts of relatively pure proteins with full biological activity (deLisle et al., Techniques in Protein Chemistry IV. Academic Press, New York, pp. 257-267 (1992)).

Recombinant techniques can also be used to modify nanobodies thereof. For example, amino acids found to not contribute to either the activity or the binding specificity or affinity of the nanobody can be deleted without a loss in the respective activity. Insertions, deletions, substitutions, or other selected modifications of particular regions or specific amino acids residues can also be made (and are contemplated by the present disclosure), provided the activity of the fragment is not significantly altered or impaired compared to the non-modified nanobody, or antigen binding fragment thereof can be made. Such methods are readily apparent to a skilled practitioner in the art and can include site specific mutagenesis of the nucleic acid encoding the nanobody or fragment thereof. (Zoller et al., Nucl. Acids Res. 10:6487-500 (1982)).

Following expression, the nanobodies can be isolated. A nanobody can be isolated or purified in a variety of ways known in the art depending on what other components are present in the sample. Standard purification methods include electrophoretic, molecular, immunological, and chromatographic techniques, including ion exchange, hydrophobic, affinity, and reverse-phase HPLC chromatography. For example, a nanobody can be purified using a standard anti-antibody column (e.g., a protein-A or protein-G column that can be adapted for nanobodies). Ultrafiltration and diafiltration techniques, in conjunction with protein concentration, are also useful. See, e.g., Scopes (1994) Protein Purification, 3^{rd} edition, Springer-Verlag, New York City, New York. The degree of purification necessary varies depending on the desired use. In some instances, no purification of the expressed nanobody or fragments thereof is necessary.

Methods for determining the yield or purity of a purified nanobody or fragment thereof are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

iii. Modification of Nanobodies

Any of the CALR-binding nanobodies described herein can be modified. The modifications can be covalent or non-covalent modifications. Such modifications can be introduced into the nanobodies by, e.g., reacting targeted amino acid residues of the polypeptide with an organic derivatizing agent that is capable of reacting with selected side chains or terminal residues. Suitable sites for modification can be chosen using any of a variety of criteria including, e.g., structural analysis or amino acid sequence analysis of the nanobodies or fragments. In some instances, the nanobodies may be labeled by a variety of means for use in diagnostic and/or pharmaceutical applications.

In some embodiments, the nanobodies can be conjugated to a heterologous moiety. The heterologous moiety can be, e.g., a heterologous polypeptide, a therapeutic agent (e.g., a toxin or a drug), or a detectable label such as, but not limited to, a radioactive label, an enzymatic label, a fluorescent label, a heavy metal label, a luminescent label, or an affinity tag such as biotin or streptavidin. Suitable heterologous polypeptides include, e.g., an antigenic tag (e.g., FLAG (DYKDDDDK) (SEQ ID NO:16), polyhistidine (6-His; HHHHHH (SEQ ID NO:17)), hemagglutinin (HA; YPYDVPDYA (SEQ ID NO:18)), glutathione-S-transferase (GST), or maltose-binding protein (MBP)) for use in purifying the nanobodies or fragments. Heterologous polypeptides also include polypeptides (e.g., enzymes) that are useful as diagnostic or detectable markers, for example, luciferase, a fluorescent protein (e.g., green fluorescent protein (GFP)), or chloramphenicol acetyl transferase (CAT). Suitable radioactive labels include, e.g., ³²P, ³³P, ¹⁴C, ¹²⁵I, ¹³¹I, ³⁵S, and ³H. Suitable fluorescent labels include, without limitation, fluorescein, fluorescein isothiocyanate (FITC), green fluorescent protein (GFP), DyLight™ 488, phycoerythrin (PE), propidium iodide (PI), PerCP, PE-Alexa Fluor®700, Cy5, allophycocyanin, and Cy7. Luminescent labels include, e.g., any of a variety of luminescent lanthanide (e.g., europium or terbium) chelates. For example, suitable europium chelates include the europium chelate of diethylene triamine pentaacetic acid (DTPA) or tetraazacyclododecane-1,4,7,10-tetraacetic acid (DOTA). Enzymatic labels include, e.g., alkaline phosphatase, CAT, luciferase, and horseradish peroxidase. Another labeling technique which may result in greater sensitivity consists of coupling the nanobodies to low molecular weight haptens. These haptens can then be specifically altered by means of a second reaction. For example, it is common to use haptens such as biotin, which reacts with avidin, or dinitrophenol, pyridoxal, or fluorescein, which can react with specific anti-hapten nanobodies.

Two proteins (e.g., a nanobody and a heterologous moiety) can be cross-linked using any of a number of known chemical cross linkers. Examples of such cross linkers are those that link two amino acid residues via a linkage that includes a “hindered” disulfide bond. In these linkages, a disulfide bond within the cross-linking unit is protected (by hindering groups on either side of the disulfide bond) from reduction by the action, for example, of reduced glutathione or the enzyme disulfide reductase. One suitable reagent, 4-succinimidyloxycarbonyl-α-methyl-α(2-pyridyldithio) toluene (SMPT), forms such a linkage between two proteins utilizing a terminal lysine on one of the proteins and a terminal cysteine on the other. Heterobifunctional reagents that cross-link by a different coupling moiety on each protein can also be used. Other useful cross-linkers include, without limitation, reagents which link two amino groups (e.g., N-5-azido-2-nitrobenzoyloxysuccinimide), two sulfhydryl groups (e.g., 1,4-bis-maleimidobutane), an amino group and a sulfhydryl group (e.g., m-maleimidobenzoyl-N-hydroxysuccinimide ester), an amino group and a carboxyl group (e.g., 4-[p-azidosalicylamido]butylamine), and an amino group and a guanidinium group that is present in the side chain of arginine (e.g., p-azidophenyl glyoxal monohydrate).

In some embodiments, a radioactive label can be directly conjugated to the amino acid backbone of the nanobody. Alternatively, the radioactive label can be included as part of a larger molecule (e.g., ¹²⁵I in meta-[¹²⁵I]iodophenyl-N-hydroxysuccinimide ([¹²⁵I]mIPNHS), which binds to free amino groups to form meta-iodophenyl (mIP) derivatives of relevant proteins (see, e.g., Rogers et al. (1997) J Nucl Med 38:1221-1229) or chelate (e.g., to DOTA or DTPA), which is in turn bound to the protein backbone. Methods of conjugating the radioactive labels or larger molecules/chelates containing them to the nanobodies described herein are known in the art. Such methods involve incubating the proteins with the radioactive label under conditions (e.g., pH, salt concentration, and/or temperature) that facilitate binding of the radioactive label or chelate to the protein (see, e.g., U.S. Pat. No. 6,001,329).

Methods for conjugating a fluorescent label (sometimes referred to as a fluorophore) to a protein (e.g., a nanobody) are known in the art of protein chemistry. For example, fluorophores can be conjugated to free amino groups (e.g., of lysines) or sulfhydryl groups (e.g., cysteines) of proteins using succinimidyl (NHS) ester or tetrafluorophenyl (TFP) ester moieties attached to the fluorophores. In some embodiments, the fluorophores can be conjugated to a heterobifunctional cross-linker moiety such as sulfo-SMCC. Suitable conjugation methods involve incubating a nanobody protein or fragment thereof with the fluorophore under conditions that facilitate binding of the fluorophore to the protein. See, e.g., Welch and Redvanly (2003) Handbook of Radiopharmaceuticals: Radiochemistry and Applications, John Wiley and Sons.

In some embodiments, the nanobodies can be modified, e.g., with a moiety that improves the stabilization and/or retention of the nanobodies in circulation, e.g., in blood, serum, or other tissues. For example, the nanobody can be PEGylated as described in, e.g., Lee et al. (1999) Bioconjug Chem 10(6): 973-8; Kinstler et al. (2002) Advanced Drug Deliveries Reviews 54:477-485; and Roberts et al. (2002) Advanced Drug Delivery Reviews 54:459-476, or HESylated (Fresenius Kabi, Germany) (see, e.g., Pavisid et al. (2010) Int J Pharm 387(1-2):110-119). The stabilization moiety can improve the stability, or retention of, the nanobody (or fragment) by at least 1.5 (e.g., at least 2, 5, 10, 15, 20, 25, 30, 40, or 50 or more) fold.

In some embodiments, the nanobodies described herein can be glycosylated. In some embodiments, a nanobody described herein can be subjected to enzymatic or chemical treatment, or produced from a cell, such that the nanobody has reduced or absent glycosylation. Methods for producing nanobodies with reduced glycosylation are known in the art and described in, e.g., U.S. Pat. No. 6,933,368; Wright et al. (1991) EMBO J 10(10):2717-2723; and Co et al. (1993) Mol Immunol 30:1361, and can be applied to nanobodies.

iv. Imaging Agents, Chelating Agents, and Linkers

As described above, nanobodies and antigen-binding fragments thereof can be modified, for example, for use as probes or imaging probes. According to Formula 1 or Formula 2 above, X comprises an anti-calreticulin nanobody or antigen-binding fragment thereof. In embodiments, an anti-calreticulin nanobody can comprise a fluorochrome, caged fluorochrome, or radioisotope; wherein B is an optional linker and other aspects as described below.

In some embodiments, A comprises one or more imaging agents selected from the group consisting of a paramagnetic ion, an x-ray imaging agent, a fluorophore, and a radioisotope. In some embodiments, A comprises 1, 2, or 3 imaging agents selected from the group consisting of a paramagnetic ion, an x-ray imaging agent, a fluorophore, and a radioisotope. In some embodiments, A comprises one imaging agent. In some embodiments, A comprises two imaging agents. In some embodiments, A comprises three imaging agents.

In some embodiments, A comprises one or more imaging agents which can include one or more independently selected paramagnetic ions. In some embodiments, each of the paramagnetic ions are independently selected from the group consisting of chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), and erbium (III). In some embodiments, A comprises 1, 2, or 3 independently selected paramagnetic ions. In some embodiments, the one or more independently paramagnetic ions are independently directly or indirectly (e.g., through a chelator) bound to the compounds provided herein.

In some embodiments, A comprises one or more imaging agents which are independently selected x-ray imaging agents which are independently selected x-ray imaging agents. In some embodiments, each of the x-ray imaging agents are independently selected from the group consisting of lanthanum (III), gold (III), lead (II), and bismuth (III). In some embodiments, A comprises 1, 2, or 3 independently selected x-ray imaging agents.

In some embodiments, A comprises one or more imaging agents which include one or more independently selected radioisotopes. In some embodiments, each of the radioisotopes are independently selected from the group consisting of ³H, ¹¹C, ¹⁴C, ¹⁸F, ³²P, ³⁵S, ³⁶Cl, ⁵¹Cr, ⁵²Fe, ⁵⁷Co, ⁵⁸Co, ⁵⁹Fe, ⁶⁴Cu, ⁶⁷Cu, ⁶⁷Ga, ⁶⁸Ga, ⁷⁵Se, ⁷Br, ⁷⁷Br, Zr⁸⁹, ⁹⁰Y, ^{99m}Tc, ¹¹¹In, ¹²³I, ¹²⁴I, ¹²⁵I, ¹³¹I, ¹⁵²Eu, ¹⁵³Sm, ¹⁶⁶Ho, ¹⁷⁷Lu, ¹⁸⁶Re, ¹⁸⁸Re, ²⁰¹TI, ²⁰³Pb, ²¹⁰At, ²¹¹At, ²¹²Bi, ²¹³Bi, and ²²⁵Ac. In some embodiments, the one or more independently radioisotopes are independently directly or indirectly (e.g., through a chelator) bound to the compounds provided herein.

In some embodiments, A is an imaging agent selected from the group consisting of a positron emission tomography (PET) imaging agent, a single-photon emission computed tomography (SPECT) imaging agent, and a computed tomography imaging agent. In some embodiments, A is a PET or SPECT imaging agent. In some embodiments, A is a PET imaging agent. In some embodiments, A is a SPECT imaging agent. In some embodiments, A is a computed tomography imaging agent. In some embodiments, A is a radioisotopic computed tomography imaging agent.

In some embodiments, A is a PET or SPECT imaging agent comprising one or more radioisotopes selected from the group consisting of ³H, ¹¹C, ¹⁴C, ¹⁸F, ³²P, ³⁵S, ⁵²Fe, ⁵⁸Co, ⁶⁴Cu, ⁶⁸Ga, ⁷⁶Br, ⁷⁷Br, ⁸⁹Zr, ¹¹¹In, ¹²³I, ¹²⁴I, ¹²⁵I, ¹³¹I, ¹⁸⁶Re, ¹⁸⁸Re, and ²⁰¹TI,. In some embodiments, A is a PET or SPECT imaging agent comprising 1, 2, or 3 radioisotopes selected from the group consisting of ³H, ¹¹C, ¹⁴C, ¹⁸F, ³²P, ³⁵S, ⁵²Fe, ⁵⁸Co, ⁶⁴Cu, ⁶⁸Ga, ⁷⁶Br, ⁷⁷Br, ⁸⁹Zr, ¹¹¹In, ¹²³I, ¹²⁴I, ¹²⁵I, ¹³¹I, ¹⁸⁶Re, ¹⁸⁸Re, and ²⁰¹TI. In some embodiments, A is a PET or SPECT imaging agent comprising one radioisotope selected from the group consisting of ³H, ¹¹C, ¹⁴C, ¹⁸F, ³²P, ³⁵S, ⁵²Fe, ⁵⁸Co, ⁶⁴Cu, ⁶⁸Ga, ⁷⁶Br, ⁷⁷Br, ⁸⁹Zr, ¹¹¹In, ¹²³I, ¹²⁴I, ¹²⁵I, ¹³¹I, ¹⁸⁶Re, ¹⁸⁸Re, and ²⁰¹TI. In some embodiments, A is a PET or SPECT imaging agent comprising ⁶⁸Ga. In embodiments, the radioisotope can be Radium-223 or Technetium-99m.

In some embodiments, A further comprises a cheating agent. Example chelating agents include, but are not limited to, 1,4,7-triazacyclononanetriacetic acid (NOTA), 1,4,7,10-tetraazacyclododecane-1,4,7,10-tetraacetic acid (DOTA), 1,4,7-triazacyclononane-1-glutaric acid-4,7-diacetic acid (NODAGA), ethylene diamine tetra-acetic acid (EDTA), diethylene triaminepentaacetic acid (DTPA), cyclohexyl-1,2-diaminetetraacetic acid (CDTA), ethyleneglycol-O,O′-bis(2-aminoethyl)-N,N,N′,N′-tetraacetic acid (EGTA), N,N-bis(hydroxybenzyl)-ethylenediamine-N,N′-diacetic acid (HBED), triethylene tetramine hexaacetic acid (TTHA), hydroxyethyidiamine triacetic acid (HEDTA), and 1,4,8,11-tetraazacyclotetradecane-N,N′,N″,N′″-tetraacetic acid (TETA), TCMC, and DFO. In some embodiments, the chelating agent is selected from the group consisting of 1,4,7-triazacyclononanetriacetic acid (NOTA), 1,4,7,10-tetraazacyclododecane-1,4,7,10-tetraacetic acid (DOTA), 1,4,7-triazacyclononane-1-glutaric acid-4,7-diacetic acid (NODAGA), ethylene diamine tetra-acetic acid (EDTA), diethylene triaminepentaacetic acid (DTPA), cyclohexyl-1,2-diaminetetraacetic acid (CDTA), ethyleneglycol-O,O′-bis(2-aminoethyl)-N,N,N′,N′-tetraacetic acid (EGTA), N,N-bis(hydroxybenzyl)-ethylenediamine-N,N′-diacetic acid (HBED), triethylene tetramine hexaacetic acid (TTHA), hydroxyethyidiamine triacetic acid (HEDTA), and 1,4,8,11-tetraazacyclotetradecane-N,N′,N″,N′″-tetraacetic acid (TETA), TCMC, and DFO. In some embodiments, the chelating agent is selected from the group consisting of 1,4,7-triazacyclononanetriacetic acid (NOTA), 1,4,7,10-tetraazacyclododecane-1,4,7,10-tetraacetic acid (DOTA), and 1,4,7-triazacyclononane-1-glutaric acid-4,7-diacetic acid (NODAGA). In some embodiments, the chelating agent is 1,4,7-triazacyclononanetriacetic acid (NOTA). In some embodiments, A further comprises one or more chelating agents. In some embodiments, A further comprises 1, 2, or 3 chelating agents. In some embodiments, A further comprises one chelating agent. In some embodiments, A further comprises two chelating agents. In some embodiments, A further comprises three chelating agents. In embodiments, the chelator is NOTA-Benzyl-NCS.

In some embodiments, A comprises one or more imaging agents which are independently selected fluorophores. Example fluorophores include, but are not limited to Alexa 350, Alexa 430, AMCA, BODIPY 630/650, BODIPY 650/665, BODIPY-FL, BODPY-R6G, 13BODLPY-TMR, BODLPY-TRX, cascade blue, Cy3, Cy5, 6-FAM, fluorescein isothiocyanate, HEX, 6-JOE, oregon green 488, oregon green 500, oregon green 514, quantum dots, pacific blue, REG, rhodamine green, rhodamine red, renographin, ROX, TAMRA, TET, tetramethylrhodamine, Texas Red, the Alexafluor family, Cy5, Cy5.5, Cy7, indocyanine green (ICG), and fluorescent proteins (e.g., green fluorescent protein (GFP), red fluorescent protein (RFP), and dsRED).

In emobdiments, the fluorophore can be a constitutively fluorescing fluorochrome or a caged fluorophore (i.e., one that requires a stimulus to induce fluorescence).

In embodiments, the fluorophore can be a near-infrared (NIR) cyanine dye, a hemicyanine dye, a coumarin-based dye, a fluorescein-based dye, a rhodamine-based dye, a BODIPY-Based Dye, a BODIPY-Based NIR Dye, a Xanthene-Based dye, a Xanthene-Based NIR Dye, a cyanine-based dyes, a porphyrin dye, a phthalocyanine dye, a squaraine dye, or any combination of any thereof.

In embodiments, the fluorophore comprises Cy5.5, Cy7, IRDye8000W, a Silicon Rhodamine, a Janelia Fluor 646, a Janelia Fluor 669, a Janelia Fluor 774); Squaraine Dyes, ICG, or IR-800, individually or in any combination of any thereof.

In some embodiments, A is a fluorophore selected from the group consisting of Alexa 350, Alexa 430, AMCA, BODIPY 630/650, BODIPY 650/665, BODIPY-FL, BODPY-R6G, 13BODLPY-TMR, BODLPY-TRX, cascade blue, Cy3, Cy5, 6-FAM, fluorescein isothiocyanate, HEX, 6-JOE, oregon green 488, oregon green 500, oregon green 514, quantum dots, pacific blue, REG, rhodamine green, rhodamine red, renographin, ROX, TAMRA, TET, tetramethylrhodamine, Texas Red, AF 350, 405, AF532, AF488, AF647, AF680, AF750, Cy5, Cy5.5, Cy7, indocyanine green (ICG), green fluorescent protein (GFP), red fluorescent protein (RFP), and dsRED. In some embodiments, A comprises 1, 2, or 3 independently selected fluorophores.

In some embodiments, B is an optional linking group comprising one or more amino acid residues. In some embodiments, B is an optional linking group comprising about 1 to about 100 amino acid residues, for example about 1 to about 100, about 1 to about 80, about 1 to about 60, about 1 to about 40, about 1 to about 20, about 1 to about 10, about 1 to about 5, about 5 to about 100, about 5 to about 80, about 5 to about 60, about 5 to about 40, about 5 to about 20, about 5 to about 10, about 10 to about 100, about 10 to about 80, about 10 to about 60, about 10 to about 40, about 10 to about 20, about 20 to about 100, about 20 to about 80, about 20 to about 60, about 20 to about 40, about 40 to about 100, about 40 to about 80, about 40 to about 60, about 60 to about 100, about 60 to about 80, or about 80 to about 100 amino acid residues.

In some embodiments, B is an optional linking group comprising one or more alkylene groups, one or more amine groups, one or more amide groups, one or more alkyleneoxy groups, one or more thiol groups, one or more carbohydrate groups, or any combination thereof. In some embodiments, B is an optional linking group comprising one or more alkylene groups, one or more amine groups, one or more amide groups, one or more alkyleneoxy groups, one or more thiol groups, or any combination thereof. In some embodiments, B is an optional linking group comprising one or more C₁₋₅₀ alkylene groups, one or more amine groups, one or more amide groups, one or more C₁₋₅₀ alkyleneoxy groups, one or more C₁₋₅₀ thiol groups, or any combination thereof. In some embodiments, B is an optional linking group comprising one or more C₁₋₃₀ alkylene groups, one or more amine groups, one or more amide groups, one or more C₁₋₃₀ alkyleneoxy groups, one or more C₁₋₃₀ thiol groups, or any combination thereof.

In some embodiments, B is an optional linking group comprising one or more —(OCH₂CH₂)— groups. In some embodiments, B is an optional linking group of the formula —(OCH₂CH₂)ₚ—, wherein p is an integer from about 1 to about 100, for example, from about 1 to about 100, about 1 to about 80, about 1 to about 60, about 1 to about 40, about 1 to about 20, about 1 to about 10, about 1 to about 5, about 5 to about 100, about 5 to about 80, about 5 to about 60, about 5 to about 40, about 5 to about 20, about 5 to about 10, about 10 to about 100, about 10 to about 80, about 10 to about 60, about 10 to about 40, about 10 to about 20, about 20 to about 100, about 20 to about 80, about 20 to about 60, about 20 to about 40, about 40 to about 100, about 40 to about 80, about 40 to about 60, about 60 to about 100, about 60 to about 80, or about 80 to about 100 amino acid residues. In some embodiments, p is an integer from about 10 to about 40. In some embodiments, p is an integer from about 20 to about 40. In some embodiments, p is an integer from about 25 to about 35.

Embodiments of radioisotope-tagged nanobodies can be found, for example, in Example 1 below.

v. Polynucleotides Encoding CALR-Binding Nanobodies

Also described herein are polynucleotides encoding CALR-binding nanobodies, as well as expression vectors comprising a polynucleotide described herein. Such polynucleotides and vectors described herein can be utilized, for example and without intending to be limiting, for nanobody production and/or administration into a cell.

### B. Synthesis of Calreticulin Nanobodies

Synthesis of nanobodies according to the present disclosure can be carried out, for example, by utilizing the synthetic routes described further in detail in the Figures and Examples below.

### II. KITS AND PACKAGING

The calreticulin (CALR)-targeting nanobodies and/or compositions (i.e., those comprising, consisting essentially of, or consisting of one or more calreticulin (CALR)-targeting nanobodies described herein) can be utilized in the preparation of a kit. In some embodiments, kits are provided for carrying out any of the methods described herein. The kits of this disclosure may comprise a carrier container being compartmentalized to receive in close confinement one or more containers such as vials, tubes, and the like, each of the containers comprising one of the separate elements to be used in the method.

In some instances, one of the containers may comprise a composition as described in this disclosure that is, or can be, detectably labeled. The kit may also have containers containing buffer(s) and/or a container comprising a reporter-means, such as a biotin-binding protein, such as avidin or streptavidin, bound to a reporter molecule, such as an enzymatic or fluorescent label. In some embodiments, the kit comprises separate containers containing compositions described herein and a detectable label.

A composition as described in this disclosure for use in determining CALR activity (i.e., translocation to the cellular membrane) in a sample from a subject as described herein may be delivered in a pharmaceutical package or kit to doctors and cancer patients. Such packaging is intended to aid healthcare professionals with diagnostic ability and/or improve patient convenience and compliance with the diagnostic and/or treatment plan. Typically, the packaging comprises paper (cardboard) or plastic. In some embodiments, the kit or pharmaceutical package further comprises instructions for use (e.g., for administering according to a method as described herein).

In some embodiments, a pharmaceutical package or kit comprises unit dose forms of a composition or components of compositions described herein. In some embodiments, the pharmaceutical package or kit further comprises unit dose forms of one or more of an additional therapeutic, for example, another medicament used for treatment of a disorder in a patient. The dose unit form can be a diagnostic unit dose form.

In one embodiment, the kit or pharmaceutical package comprises a composition as described herein in a defined, therapeutically effective dose in a single unit dosage form or as separate unit doses. The dose and form of the unit dose (e.g., lyophilized, reconstituted, aqueous, tablet, capsule, immediate release, delayed release, etc.) can be any doses or forms as described herein.

In certain embodiments, kits are provided for producing a single-dose administration unit, in particular for in vivo administration. In certain embodiments, kits containing single or multi-chambered pre-filled syringes are included. In certain embodiments, kits containing one or more containers of a formulation described in this disclosure are included.

### III. METHODS OF USE AND TREATMENT

In embodiments, nanobodies according to the present disclosure can be administered to a subject or sample for the purposes or diagnosis or therapeutics. In embodiments, probes according to the present disclosure can be utilized in methods of imaging or otherwise diagnosing tumors, in particular, with PET imaging. In certain aspects, probes described herein can be utilized for targeting a given tumor in human subject for imaging purposes or targeting a tumor type in human subject for delivery of a conjugated therapeutic modality (e.g., radiation or small molecule). Probes can be given systemically (e.g., intravenously) or locally (e.g., direct injection, local instillation, or regional perfusional therapy). Probes as described herein can also be utilized as a diagnostic agent in an immuno-based assay (e.g., ELISA).

In embodiments, a method of using a calreticulin-targeting nanobody is described, comprising contacting a sample with a calreticulin-targeting nanobody as described herein (or composition comprising such as described herein); incubating the contacted sample at a temperature for a period of time; and imaging the incubated sample to determine a nanobody level.

In embodiments, sample is a tumor resection from a tumor of a subject or a tumor or suspected tumor in a subject.

In embodiments, the subject is a subject undergoing or suspected to undergo a therapy regime for treating a cancer.

In embodiments, the tumor or cancer according to the methods of the present disclosure is: an adenocarcinoma, an esophageal cancer, a gastric cancer, a small bowel cancer, a colon cancer, a rectal cancer, an anal cancer, a lunch cancer, a mesothelioma, a peritoneal cancer, an ovarian cancer, a uterine cancer, a breast cancer, a renal cancer, a urothelial cancer, a prostate cancer, a hepatoceullar carcinoma, a cholangiocarcinoma, a lymphoma, a leukemia, a liposarcoma, a, rhabdosarcoma, a fibrosarcoma, an angiosarcoma, a chondrosarcoma, a thyroid cancer, an oral cancer, a head and neck cancer, or a glioblastoma.

The compositions described herein are useful in, inter alia, imaging or otherwise diagnosing a tumor. As used herein, the term subject means a mammalian subject. Exemplary subjects include, but are not limited to humans, monkeys, dogs, cats, mice, rats, cows, horses, camels, goats and sheep. In some embodiments, the subject is a human. In some embodiments, the subject has or is suspected to have a cancer.

As used herein, administer or administration refers to the act of injecting or otherwise physically delivering a substance as it exists outside the body into a subject or patient, such as by mucosal, intradermal, intravenous, intramuscular, subcutaneous delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof. In particular, compositions comprising probes as described herein may be administered to a subject, for example, during a tumor resection surgery to help define the boundaries of any one or more tumors as described herein.

The compositions can be administered to a subject, e.g., a murine or human subject, using a variety of methods that depend, in part, on the route of administration. The route can be, e.g., intravenous injection or infusion (IV), subcutaneous injection (SC), intraperitoneal (IP) injection, intramuscular injection (IM), intradermal injection (ID), subcutaneous, transdermal, intracavity, oral, intracranial injection, or intrathecal injection (IT). The injection can be in a bolus or a continuous infusion. Techniques for preparing injectate or infusate delivery systems containing nanobodies are well known to those of skill in the art. Generally, such systems should utilize components which will not significantly impair the biological properties of the nanobodies, such as the paratope binding capacity (see, for example, Remington's Pharmaceutical Sciences, 18th edition, 1990, Mack Publishing). Those of skill in the art can readily determine the various parameters and conditions for producing nanobody injectates or infusates without resort to undue experimentation. In some embodiments, compositions as described herein can be therapeutically delivered to a subject by way of intravenous administration.

Administration can be achieved by, e.g., topical administration, local infusion, injection, and the like. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media (phosphate-buffered saline, for example). Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and the like. Pharmaceutically-acceptable carriers can be carriers suitable for administering to a patient or a subject in a clinical setting.

### IV. COMPOSITIONS, PHARMACEUTICAL FORMULATIONS, AND ROUTES OF ADMINISTRATION

Embodiments of the present disclosure include one or more active agents (i.e., one or more nanobodies or antigen-binding fragments described herein, alone or part of a nanobody of Formula 1 or 2) and formulated with one or more excipients, diluents, carriers and/or adjuvants (pharmaceutically acceptable or non-pharmaceutically acceptable for research). In addition, embodiments of the present disclosure include one or more active agents formulated with one or more pharmaceutically acceptable auxiliary substances. In particular the one or more active agents can be formulated with one or more pharmaceutically acceptable excipients, diluents, carriers, and/or adjuvants to provide an embodiment of a composition of the present disclosure.

A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H.C. Ansel et al., eds., 7^{th} ed., Lippincott, Williams, & Wilkins; and Handbook of Pharmaceutical Excipients (2000) A.H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In an embodiment of the present disclosure, the one or more active agents can be administered to the subject using any means capable of resulting in the desired effect. Thus, the one or more active agents can be incorporated into a variety of formulations for therapeutic administration. For example, the one or more active agents can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the one or more active agents may be administered in the form of its pharmaceutically acceptable salts, or a subject active composition may be used alone or in appropriate association, as well as in combination, with other pharmaceutically active compounds. The following methods and excipients are merely exemplary and are in no way limiting.

Embodiments of the one or more active agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compositions. Similarly, unit dosage forms for injection or intravenous administration may comprise the one or more active agents in a composition as a solution in sterile water, normal saline or another pharmaceutically acceptable carrier.

Embodiments of the one or more active agents can be formulated in an injectable composition in accordance with the disclosure. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles in accordance with the present disclosure.

In an embodiment, the one or more active agents can be formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of the one or more active agents can be accomplished using any of a variety of refillable, pump systems. Pumps provide consistent, controlled release over time. In some embodiments, the one or more active agents can be in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, an active agent (e.g., a nanobody or fragment thereof) can be delivered using an implantable drug delivery system, e.g., a system that is programmable to provide for administration of the agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present disclosure is the Synchromed infusion pump (Medtronic).

Suitable excipient vehicles for the one or more active agents are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pennsylvania, 17th edition, 1985. The composition or formulation to be administered will, in any event, contain a quantity of the one or more active agents adequate to achieve the desired state in the subject being treated.

Compositions of the present disclosure can include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, a pharmaceutical composition of the present disclosure (as well as combination compositions) can be delivered in a controlled release system. For example, the one or more active agents may be administered using intravenous infusion, an implantable osmotic pump, a transdermal patch, liposomes, or other modes of administration. In one embodiment, a pump may be used (Sefton (1987). CRC Crit. Ref. Biomed. Eng. 14:201; Buchwald et al. (1980). Surgery 88:507; Saudek et al. (1989). N. Engl. J. Med. 321:574). In another embodiment, polymeric materials are used. In yet another embodiment a controlled release system is placed in proximity of the therapeutic target thus requiring only a fraction of the systemic dose. In yet another embodiment, a controlled release system is placed in proximity of the therapeutic target, thus requiring only a fraction of the systemic. Other controlled release systems are discussed in the review by Langer (1990). Science 249:1527-1533.

In another embodiment, the compositions of the present disclosure (as well as combination compositions separately or together) include those formed by impregnation of the one or more active agents described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

### A. Dosages

Embodiments of the one or more active agents can be administered to a subject in one or more doses. Those of skill will readily appreciate that dose levels can vary as a function of the specific the one or more active agents administered, the severity of the symptoms and the susceptibility of the subject to side effects. Preferred dosages for a given one or more active agents are readily determinable by those of skill in the art by a variety of means.

In an embodiment, multiple doses of the one or more active agents are administered. The frequency of administration of the one or more active agents can vary depending on any of a variety of factors, e.g., severity of the symptoms, and the like. For example, in an embodiment, the one or more active agents can be administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). As discussed above, in an embodiment, the one or more active agents is administered continuously.

The duration of administration of the one or more active agents, e.g., the period of time over which the one or more active agents is administered, can vary, depending on any of a variety of factors, e.g., patient response, etc. For example, the one or more active agents in combination or separately, can be administered over a period of time of about one day to one week, about two weeks to four weeks, about one month to two months, about two months to four months, about four months to six months, about six months to eight months, about eight months to 1 year, about 1 year to 2 years, or about 2 years to 4 years, or more.

### B. Routes of Administration

Embodiments of the present disclosure provide methods and compositions for the administration of the one or more active agents to a subject (e.g., a human) using any available method and route suitable for drug delivery, including in vivo and ex vivo methods, as well as systemic and localized routes of administration.

Routes of administration include intranasal, intramuscular, intratracheal, subcutaneous, intradermal, topical application, intravenous, rectal, nasal, oral, and other enteral and parenteral routes of administration. Routes of administration may be combined, if desired, or adjusted depending upon the agent and/or the desired effect. One or more active agents can be administered in a single dose or in multiple doses. In certain aspects, the delivery may be rectal and in certain aspects may be through a suppository.

Embodiments of the one or more active agents can be administered to a subject using available conventional methods and routes suitable for delivery of conventional drugs, including systemic or localized routes. In general, routes of administration contemplated by the disclosure include, but are not limited to, enteral, parenteral, or inhalational routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of the one or more active agents. Where systemic delivery is desired, administration typically involves invasive or systemically absorbed topical or mucosal administration of pharmaceutical preparations.

In an embodiment, the one or more active agents can also be delivered to the subject by enteral administration. Enteral routes of administration include, but are not limited to, oral and rectal (e.g., using a suppository) delivery.

Methods of administration of the one or more active agents through the skin or mucosa include, but are not limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

Other features, objects, and advantages of the present invention are apparent in the description that follows. It should be understood, however, that the description, while exemplifying certain embodiments of the present invention, is given by way of illustration only, not limitation. Various changes and modifications within the scope of the invention will become apparent to those skilled in the art from the detailed description.

### V. EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in °C, and pressure is in atmosphere. Standard temperature and pressure are defined as 25 °C and 1 atmosphere.

### Example 1

Nanobodies, or single domain antibodies, are versatile molecules with therapeutic and diagnostic applications, but they are primarily developed by panning nanobody libraries isolated from immunized camelids. This approach is not effective for non-immunogenic or toxic antigens, prevents the use of scaffolds with ideal pharmacokinetic properties, and each target antigen requires its own unique immunization campaign. To circumvent these limitations, a modular synthetic nanobody phage display vector was developed based on the scaffold of the 2Rs15d nanobody that allows for the manipulation of the binding and framework regions of the nanobody. Using this vector, a library of nanobodies was created with a randomized CDR2 with over 1.7×10⁶ unique sequences/μL. As a proof-of-concept, the library was panned for nanobodies binding calreticulin (CALR), a protein critical in immunogenic cell death. One isolated clone, Cal3, has a measured affinity of 37 nM for CALR and is cross-reactive with mouse and human CALR. Using positron emission tomography (PET) imaging, the radiolabeled ⁶⁴Cu-NOTA-Cal3 is demonstrated to detect changes in tumor CALR expression in vivo, the first reported synthetic nanobody characterized by PET imaging. This study demonstrates the feasibility of building and panning of synthetic libraries for high affinity nanobodies as an alternative to immunized camelid libraries. Looking forward, not only can this modular synthetic nanobody phage display vector can be used to generate novel nanobodies by randomizing its binding regions, but the framework regions can also be manipulated to select for nanobodies with ideal pharmacokinetic and production properties.

### A. Introduction

Based on the advantages outlined herein, here it is reported the development of a novel synthetic nanobody phage display library. The framework of this library is based on the 2Rs15d nanobody, a well characterized nanobody that is currently in clinical trials for PET imaging [8, 9]. The 2Rs15d nanobody, initially identified in 2011 by VanEckyen et al. through biopanning a HER2 immunized camelid phage display library, has gained widespread recognition [10]. It has been researched both in preclinical and clinical contexts, serving as a PET imaging agent for HER2+ cancers and under evaluation for targeted radiotherapy [11-14]. Other studies have actively characterized the 2Rs15d nanobody by conducting a structural analysis of its interaction with HER2, investigating its in vivo distribution and clearance, and evaluating its binding affinity and kinetics [13, 15, 16].

As demonstrated by the success of 2Rs15d as a nuclear imaging agent, nanobodies are valuable PET tracers that can elucidate metabolic, phenotypic, and mechanistic characteristics of countless diseases, including cancer [2]. PET imaging is essential for both preclinical research and clinical management of patients with cancer. Rather than simply observing the morphological structures of the tumor burden that is possible with magnetic resonance imaging (MRI) and computed tomography (CT), the characteristics of the PET tracer allow for assessment of disease metabolism, receptor expression, and other disease phenotypes [17]. In short, a ligand radiolabeled with a positron-emitting radionuclide, such as ¹⁸F, ⁸⁹Zr, or ⁶⁴Cu, is injected intravenously into the subject. The ligand localizes the tracer to the tissue of interest, and the radionuclide emits a positron that collides with an electron in the tissue. The collision results in the release of two coincident photons that are detected by the PET scanner. This allows for the exact location and intensity of the signal to be measured and computed [18]. This process is noninvasive; therefore, disease progression and response can be measured longitudinally. Furthermore, PET imaging scans the entire body of the subject, allowing for the assessment of the total disease burden. While biopsy is a critical tool in the stratification of patients who may or may not respond to certain therapies, it is limited by its spatial and temporal nature. PET imaging and other nuclear imaging techniques can overcome these limitations by measuring dynamic processes and heterogeneity that contribute to patient outcomes. Therefore, the ability to develop novel nanobody-based PET tracers can revolutionize preclinical research and clinical results.

As a proof-of-concept target, selections for nanobodies specific to calreticulin (CALR) were conducted. Calreticulin, a multifunctional endoplasmic reticulum-resident protein, has gained increasing significance as a biomarker in cancer therapy [19]. Its role extends beyond its classical function in calcium homeostasis and protein folding, as emerging research suggests that altered calreticulin expression is associated with various cancers [20]. Importantly, the involvement of calreticulin in immune response modulation as a damage-associated molecular pattern (DAMP), particularly its expression during immunogenic cell death, makes it a strong candidate biomarker for interrogating the tumor microenvironment [21]. A non-invasive biomarker for calreticulin could be used to assess the effectiveness of specific therapies in inducing immunogenic cell death, act as guide for combination immunotherapies, or even serve as a cancer-specific therapeutic target. To date, only one peptide-based imaging agent has been described [22]. While this peptide has been demonstrated to detect increases in calreticulin following doxorubicin treatment, its micromolar affinity may limit clinical utility.

In this study, a synthetic nanobody library has been panned for a nanobody with affinity and specificity for calreticulin (CALR), demonstrate its cross reactivity has been demonstrated with mouse and human CALR, and its in vivo detection of CALR via PET imaging assessed.

### B. Methods

### Library Cloning

A 3+3 M13 phage display library was generated by digesting a plasmid containing the DNA sequence for a phage protein pIII/2Rs15d fusion protein using the KasI and NcoI (New Enland Biolabs) restriction enzyme cut sites and ligating a synthetic DNA fragment containing the bacterial codon optimized coding sequence for the 2Rs15d nanobody (Integrated DNA Technologies).

A PfoI restriction site 16 bases upstream of the PelB leader sequence was altered by site directed mutagenesis using a silent mutation approach to abolish recognition by the restriction enzyme. In short, back-to-back primers are designed with a mismatch for the desired region, and the plasmid is linearized with the new sequence amplified. The parent plasmid is then degraded, and the linearized vectors carrying the new sequence are ligated. The resulting novel vectors are transformed into competent E. coli and sequenced for confirmation of the mutagenesis. The cycling conditions included an initial denaturation of 30 seconds at 98 °C followed by 25 cycles of 10 seconds at 98 °C, 30 seconds at 59 °C, and 3 minutes at 72 °C. The reaction concluded with a final extension of 2 minutes at 72 °C and an infinite hold at 4 °C. The PCR reaction was comprised of 25 ng of template plasmid, 10 μM forward and reverse primers, and 1×Q5 Hot Start Master Mix (New England Biolabs) with a final reaction volume of 25 μL. Following PCR, the reaction was treated with KLD enzyme mix (New England Biolabs) and transformed into NEB 5-alpha competent E. coli (New England Biolabs). Clones were isolated and screened for successful mutagenesis by Sanger sequencing using a primer upstream of the nanobody sequence (5′ TATGACCATGATTACGCCAAGC 3′, SEQ ID NO:1).

To randomize residues, a single stranded oligonucleotide comprising of random amino acids encoded by the degenerate codon NNK, where N is any nucleotide and K is a G or T, corresponding to the position of the selected residues in the scaffold was synthesized and amplified as a double stranded fragment via Klenow synthesis (Integrated DNA Technologies). In short, 5 μg of the single-stranded insert is combined with 5× molar excess of reverse primer. The reaction was incubated at 52 °C for 10 minutes. The resulting annealed reaction was added to 1×NEB buffer #2, 15 units Klenow fragment (3′→5′ exo-) (New England Biolabs) and 0.4 mM dNTPs (New England Biolabs). The mixture was incubated at 37 °C for 30 minutes then desalted with a PCR clean up kit (New England Biolabs). The nanobody-phagemid vector was amplified in SCS110 E. coli (Agilent) incapable of methylating DNA and isolated by miniprep (New England Biolabs).

Three μg each of the unmethylated vector and randomized double-stranded insert was digested overnight at 37 °C with 3 μL FastDigest PfoI (ThermoFisher). Following the initial digestion, the products were run on a 1% agarose gel and extracted with the Monarch® DNA Gel Extraction kit (New England Biolabs). The products were incubated overnight at 37 °C with 5 units BsgI (New England Biolabs). The digested insert was desalted with a PCR clean up kit, and the digested vector was run on a 1% agarose gel and extracted as previously described. 150 ng of digested vector was ligated with a 3:1, 5:1, and 7:1 molar excess of digested insert with 1,000 units T4 DNA ligase (New England Biolabs) for 10 minutes at RT. The resulting ligation reaction was dialyzed using 0.025 μm pore size membrane filters for 4 hours. The desalted ligation reactions were transformed into Tg1 E. coli. Serial dilutions of each reaction were plated to calculate transformation efficiency. The ligated library was called Nanobody Library with randomized CDR2 (NaLiC2.2).

### Phage Selection

Biotinylated calreticulin (CALR), 50 μg lyophilized recombinant human CALR (Sino Biological) was reconstituted to 0.2 mg/mL in PBS and incubated with 10 molar excess of 10 mM EZ-Link NHC-LC-Biotin (ThermoFisher) at 37 C with shaking at 250 rpm for 1 hour. Unreacted biotin was removed by buffer exchange in an Amicon 0.5 mL 10K MWCO spin column. Biotinylated CALR (Biot-CALR) was stored in TBS to neutralize any remaining unreacted biotin. Biot-CALR concentration was determined by BCA and diluted to 0.2 mg/mL. The Pierce Biotin Quantification Kit (ThermoFisher) was used to assess biotinylation of CALR.

For round 1 of selection, 5×10¹² NaLiC2 phage blocked in 5% BSA in Tris buffered saline plus 0.1% Tween-20 (TBST). Selections wells were prepared by adsorbing 500 ng neutravidin per well in 0.1M sodium bicarbonate (pH 9.5) to Nunc Maxisorp plates for 1 hr at room temperature (RT), followed by blocking with 5% BSA in 0.1M sodium bicarbonate (pH 9.5) overnight at 4 °C. The pre-blocked library was then subtracted for neutravidin binding and nonspecific phage by incubating it in the neutravidin coated wells for 1 hr at RT with shaking at 250 rotations per minute (RPM). The subtracted library was then transferred to a microcentrifuge tube and incubated with 1 μg biotinylated CALR (Sino Biological) with shaking at 250 RPM. The phage were transferred to a neutravidin well to capture phage bound to target by shaking at 250 RPM for 1 hour at RT. The captured phage were eluted with 100 μL 0.05% trypsin-EDTA (Gibco) shaking at 37 °C, 250 rpm for 5 minutes and neutralized with 100 μL Pierce EDTA-free protease inhibitor tablet (ThermoFisher) dissolved in 10 mL TBS.

Eluted phage were titered and amplified as follows. Phage titers were determined by serially diluting phage 10-fold to 10-12 in midlog Tg1 E. coli and incubating for 30 minutes at 37 °C to allow for infection. The dilutions were plated on carbenicillin agar plates and incubated overnight at 30 °C. Colonies from each spot were counted, and phage concentrations were calculated. To amplify the eluted phage, 500 μL Tg1 E. coli at the midlog growth phase were infected with the eluted phage and incubated at 37 °C for 30 minutes. This infection was added to 5 mL LB supplemented with 2% glucose and 100 μg/mL carbenicillin and incubated overnight at 37 °C with shaking at 250 RPM. The overnight culture was added to 100 mL LB supplemented with the same concentrations of glucose and carbenicillin and grown at 37 °C with shaking at 250 RPM. 10 mL of this culture was aliquoted in and super infected with helper phage by incubating at 37 °C for 30 minutes. The phagemid was isolated from the discarded culture for deep sequencing to monitor for individual clone enrichment. The superinfected culture was pelleted and resuspended in 100 mL LB supplemented with 100 μg/mL carbenicillin, 50 μg/mL kanamycin, 1 mM IPTG, and 1 Pierce EDTA-free protease inhibitor tablet (ThermoFisher). The culture was incubated overnight at 37 °C with shaking at 250 RPM. The following day, the culture was pelleted and resuspended in 17 mL PEG/NaCl and incubated at 4 °C for at least 2 hours. The resulting mixture was centrifuged and the supernatant was discarded. The phage pellet was resuspended in 1 mL of ice-cold PBS and centrifuged again. The supernatant was transferred to a new tube and supplemented with 200 μL of ice-cold PEG/NaCl. After incubating on ice for 1 minute, the phage were pelleted again and resuspended in 200 μL of ice-cold PBS. The resuspended phage were incubated at 65 °C for 10 minutes to kill any bacterial contaminants.

For round 2, selection proceeded as in round 1 with the following exception: target binding occurred with 1 μg unbiotinylated CALR adsorbed directly to the polystyrene plate. Round 3 of selection proceeded identically to round 1.

### Sequencing and Analysis

The CDR2 of the starting library and eluted phage from each round of selection was amplified via PCR of primers flanking CDR2 (5′ TATCGGCAGAGTCCTGGAAGAGAG 3′ (SEQ ID NO:2) and 5′ GTTTCCAAGTTGTAACACACTGCAC 3′ (SEQ ID NO:3)). Cycling conditions included an initial denaturation at 98 °C for 30 seconds followed by 25 cycles of 98 °C for 10 seconds, 65 °C for 30 seconds, and 72 °C for 30 seconds. They reaction concluded with a final elongation of 2 minutes at 72 °C and an infinite hold at 4 °C. The resulting reaction was desalted using a PCR Clean Up kit (New England Biolabs). The resulting sample was submitted for next generation sequencing (NGS) at the Heflin Genomics Core at the University of Alabama at Birmingham (Birmingham, AL, USA). There it was ligated with adapters and deep sequenced with 500,000 reads per library.

A Python program connected to a MySQL database was used for analysis of NGS output. Analysis was performed by splicing the parent sequence at the first ‘N’ and last ‘K’ to create ‘heads’ and ‘tails’ sequences. The uploaded sequences were initially searched for the ‘heads’ sequence, and those that match were sorted into a separate list for searching for the ‘tails’ sequence. The sequences that had both were determined to be useful and were trimmed to retain only the CDR sequence. Following translation using a hardcoded codon table, reads per million (RPM) was calculated as a normalization parameter. This was accomplished by dividing the raw count of sequences by the total number of sequences and scaled to a million. Next, a table was created using the input experiment name with 3 columns: sequence, number of repeats, and RPM.

The analysis further involved iterating through sequences to count the occurrences of each amino acid at every position. These values were stored in dictionaries, with the position as the key and frequency as the value. Frequencies were computed by dividing the number of appearances by the total number of sequences, multiplied by the number of codons resulting in that amino acid, and then multiplied by 100 to obtain a percentage. These dictionaries, containing positions and frequencies for each amino acid, were organized into 12×20 matrices (representing 12 positions for 20 amino acids) for utilization in NumPy arrays, facilitating visualization through heatmaps.

### Nb Cloning, Expression, and Purification

The CDR2 DNA sequence (Cal1: STHVSALLYSKG (SEQ ID NO:4), Cal2: GCLAVCPHRKT (SEQ ID NO:5), Cal3: (SEQ ID NO:6)) for the selected clones was inserted into the 2Rs15d nanobody sequence. The resulting nanobody sequence was codon optimized for bacterial expression and ordered as double stranded DNA (Integrated DNA Technologies). A modified pET22b (+) vector was used for periplasmic expression of the screened nanobodies. The vector contained N-terminal PelB leader sequence. Additionally, the vector included a C-terminal FLAG and 6×HIS tags. One μg of vector and 500 ng of insert digested at 37 °C for 30 minutes with 20 units each of HindIII-HF® and NcoI-HF® (New England Biolabs). The digested vector and insert were desalted with a PCR clean up kit (New England Biolabs). 50 ng of digested vector was ligated with a 3:1, 5:1, and 7:1 molar excess of digested insert with 400 units T4 DNA ligase (New England Biolabs) for 10 minutes at RT. The resulting ligation reaction was transformed into NEB 5-alpha competent E. coli (New England Biolabs). The plasmid was isolated from spots from each ligation reaction and sequenced from the T7 promoter upstream of the nanobody. Plasmids containing successfully ligated clones were transformed into T7 Shuffle competent E. coli (New England Biolabs).

Overnight cultures of Cal3-pET22b T7 Shuffle were prepared in 10 mL LB supplemented with 100 μg/mL kanamycin sulfate and 0.5% glucose. Overnight culture was then added to 1L LB supplemented with the same concentration of carbenecillin and 0.01% glucose and incubated until OD600 reached 0.6-0.8. IPTG was added at a final concentration of 0.01 mM, and the induced cultures were grown for 16-20 hours at 23 °C with agitation at 250 RPM. Induced cultures were pelleted by centrifugation at 3,000×g for 20 minutes. Pellets were resuspended in 10 mL TES buffer (1M Tris ph8, 0.1M EDTA, 0.5M sucrose) supplemented with protease inhibitors (ThermoFisher) rocked at 4 °C for 1 hour. The solution was pelleted by centrifugation at 10,000 RPM for 15 minutes and the supernatant collected. The resulting pellet was resuspended in 10 mL 1 mM MgCl2 and rocked for 1 hour at 4 °C. The solution was centrifuged as before, and the supernatant was collected and added to the previous fraction. Protein extract was stored at 4 °C until purification.

Nanobodies were purified by immobilized metal affinity chromatography (IMAC). Protein extract was equilibrated to 10 mM Tris (pH 8), 200 mM NaCl, and 10 mM Imidazole and added to Ni-NTA Agarose (ThermoFisher) washed in binding buffer composed of 10 mM Tris (pH 8), 200 mM NaCl, 10 mM imidazole, and 0.05% TritonX. The extract-agarose mixture was rocked overnight at 4 °C. Agarose was pelleted and flow through was removed. The pellet was washed 3 times in wash buffer identical to binding buffer with 20 mM imidazole. Nbs were eluted by 3 washes with elution buffer identical to binding/wash buffer with 250 mM imidazole. Fractions containing Nb were confirmed by SDS-PAGE.

Size Exclusion Chromatography (SEC) was conducted using the GE AKTA FPLC. Nanobody elutions resulting from IMAC purification were loaded onto the Superdex Increase 75 10/300 GL column (Cytiva Life Sciences) after the column was prepared by washing with 2 column volumes (48 mL total) of High-Performance Liquid Chromatography (HPLC) grade water and one column volume (24 mL) of Tris Buffered Saline (TBS). Eluent A was HPLC-grade water and eluent B was TBS. The FPLC method is as follows: inject sample onto Superdex 75 Increased 10/300 GL column washed with TBS. Elute with 100% eluent B (TBS) for 1.5 column volumes at 0.8 mL/min flow rate. Fractions containing nanobody were identified via the UV peaks on the FPLC and verified via running SDS-PAGE gels. The nanobody-containing fractions were then buffer-exchanged using a 0.5 mL 3K spin column (Millipore) and washed with metal-free phosphate buffer saline (PBS). Nanobody concentration was determined by BCA.

### ELISA Screening

100 ng recombinant protein per well in 0.1 M sodium bicarbonate (pH 9.5) was immobilized on Maxisorp plates (ThermoFisher) and blocked with 1% milk in 0.1 M (pH 9.5) sodium bicarbonate. Nanobody dilutions from 2.5 mM to 100 nM were added to wells and incubated with shaking at RT for 1 hour. The plate was washed 15 times with 300 μL TBST. 1:1000 dilution of anti-FLAG-HRP antibody (Millipore Sigma) was added to each well and incubated for 1 hour at RT with shaking. The plate was washed 10 times with TBST. TMB (Millipore Sigma) was added to each well and covered with foil and shaken at RT for 15 minutes. Reaction was quenched with 2 M sulfuric acid and read at 450 nm on a ThermoScientific Varioskan LUX plate reader. Binding curves were calculated using GraphPad Prism 10.0.2. The curves were calculated using nonlinear regression analysis of one site total binding and one site total and nonspecific binding.

### Biolaver Interferometry (BLI) Analysis

Biolayer interferometry (BLI) was performed using an Octet RED (ForteBio) controlled by Octet DataAnalysis software (version 9.0.0.14). Streptavidin (SA) biosensors (Sartorius, 18-5020) were hydrated in TBST for at least 10 minutes before use.

For ligand immobilization, biotinylated CALR (as described above) was diluted to 10 μg/mL in TBST and loaded onto the biosensors for 600 s at 1000 rpm. After loading, biosensors were washed in TBST to remove unbound ligand. Binding kinetics were assessed by exposing the functionalized biosensors to a series of Cal3 nanobody (Nb) concentrations ranging from 23.8 nM to 22.7 μM in a 3.33-fold dilution series. Association and dissociation were monitored for 300 s each at room temperature (RT). A reference biosensor was used to correct for non-specific binding.

Data were analyzed using Octet DataAnalysis software (version 9.0.0.14), and kinetic parameters (kₐ, kd, and KD) were determined using a 1:1 binding model with global fit and steady-state analysis. Data were baseline-corrected using reference well subtraction, and negative controls included a buffer-only reference well.

### CALR Sequence Conservation Analysis

The amino acid sequences of human calreticulin (P27797) and mouse calreticulin (P14211) were obtained from UniProtKB and aligned with Smith-Waterman local alignment using Snapgene software (www.snapgene.com) [23].

### Flow Cytometry

PDAC cells (2838c3 cell line) were cultured in DMEM media supplemented with 10% fetal bovine serum and 1% penicillin-streptamycin and grown to 70% confluency, followed by treatment with either 100 nM doxorubicin or 250 μM H₂O₂ for 24 h to induce surface CALR. DMSO was used as vehicle control. After treatment, cells were harvested and stained with UV Zombie Dye (Biolegend 423108) at a 1:250 dilution as a live-dead stain. After washing, cells were incubated with Cal3 nanobody at 0.1 mg/mL for 30 minutes at 4 °C in the dark. After nanobody incubation, cells were fixed with 4% PFA, followed by incubation with a rabbit anti-FLAG primary antibody (CST #23685) at a 1:200 dilution for 30 minutes at 4 °C in the dark. An anti-rabbit-A647 secondary antibody at a 1:2450 dilution was used. Samples and appropriate compensation controls were analyzed using BD FACSymphony A5 SE channels UV446, R660, and B537.

### Bioconjugation and Radiolabeling

Nanobodies were conjugated with p-SCN-Bn-NOTA (Macrocyclics) in a 10-fold molar excess of chelator. Reaction occurred for 1 hour in metal-free sodium acetate buffer (pH 8) at 37 °C with shaking at 350 rpm. The reaction was quenched with metal-free 0.5M Tris-HCl (pH 8) and the conjugated Nbs were buffer exchanged into metal-free PBS.

⁶⁴Cu was obtained from UAB Cyclotron Facility (Birmingham, AL, USA). It was adjusted to pH 6 with metal-free 1M sodium acetate (pH 6) and 185 MBq was added to ~1 mg conjugated Nb. The reaction was incubated at 37 °C for 1 hour with shaking at 500 rpm. Free ⁶⁴Cu was removed by filtration through spin column. Radiolabeled Nb was diluted in sterile saline to 1 mCi/mL.

Radiochemical purity was assessed using radio-thin layer chromatography (radio-TLC) on silica chromatography paper (Agilent, SIG001). 1 μL (~40 kBq) of the sample was spotted and developed in 50 mM EDTA until the solvent front reached 150 mm. Radioactivity distribution was immediately measured using an AR-2000 TLC scanner (Eckert & Ziegler) equipped with a gas flow proportional detector. Retention factors (Rf) were calculated as the ratio of compound migration to solvent front migration. Radiochemical purity was determined as the percentage of radioactivity at Rf ~0.12 relative to total detected activity.

### Imaging and Analysis

The animal study protocol (IACUC-22619) was approved by the Institutional Animal Care and Use Committee of The University of Alabama at Birmingham in August 2022. Male C57/BL6 mice were subcutaneously implanted with Matrigel plugs containing either recombinant CALR or BSA at a final concentration of 3.7 μM injected on opposite shoulders immediately before radiotracer injection. Mice were injected intravenously with ~80 μCi ⁶⁴Cu-NOTA-Cal3. Under anesthesia, mice were imaged 1-hour post-injection on a Sofie GNEXT PET/CT scanner with injected intravenously 20 minutes followed by a 5-minute CT at 80 kVp. Acquired PET images were analyzed using VivoQuant (Invicro) software. Regions of interest (ROIs) were drawn using anatomic guidance of the CALR and BSA plugs, heart, kidneys, and liver. The lower ventricles of the heart were used as a measure of blood signal. The signal was used to calculate standardized uptake values (SUVs). SUVs of CALR and BSA plugs from PET imaging were assessed using unpaired t-tests (Graphpad Prism 10.0.2).

### Immunohistochemistry

Tumors were isolated from mice treated with doxorubicin as described in PET imaging studies and fixed in formalin, paraffin embedded and sliced in 5 μm sections. The slides were baked overnight at 60 °C prior to deparaffinization and hydration of tissue sections. Sections were washed in xylene and decreasing concentrations of ethanol ranging from 100% to 75%, followed by dH₂O washes. To retrieve antigen, sections were boiled in citrate buffer and cooled to room temperature. Slides were incubated in 3% H₂O₂ in TBS for 10 minutes followed by several washes with TBS. Tissue sections were outlined with a hydrophobic pen and blocked in 3% serum in 0.3% Triton X-100 in TBS for 1 hour at RT. Sections being stained with Cal3 were blocked with mouse serum, and sections being stained with a control anti-CALR Ab were blocked with goat serum. Following blocking, sections were washed with TBS. Slides were stained overnight at 4 °C with primary antibodies diluted in 3% serum in 0.3% Tween20 in TBS. Sections were stained with 0.25 μM Cal3 or 1:10,000 dilution of a control anti-CALR Ab (Sino Biological #13539-T80). Control secondary-only sections were blocked with primary antibody solution. Sections were washed several times with TBS prior to staining with secondary antibodies. Sections stained with Cal3 were incubated with an anti-FLAG-HRP conjugated Ab (Sigma-Aldrich #A8592) diluted 1:500 in primary antibody solution. Sections stained with the anti-CALR Ab were incubated with an anti-rabbit-HRP conjugated Ab (R&D Systems #HAF008) diluted 1:20 in primary antibody solution. Following incubation for 1 hour at RT, slides were washed in TBS. Sections were incubated with DAB for 15 minutes then dipped in hematoxylin solution, DI water, and incubated in tap water. Slides were dehydrated and cleared using sequential washes in increasing concentrations of ethanol ranging from 50% to 100%, followed by incubation in xylene. Slides were covered, dried overnight, and imaged at 40× magnification.

### C. Results

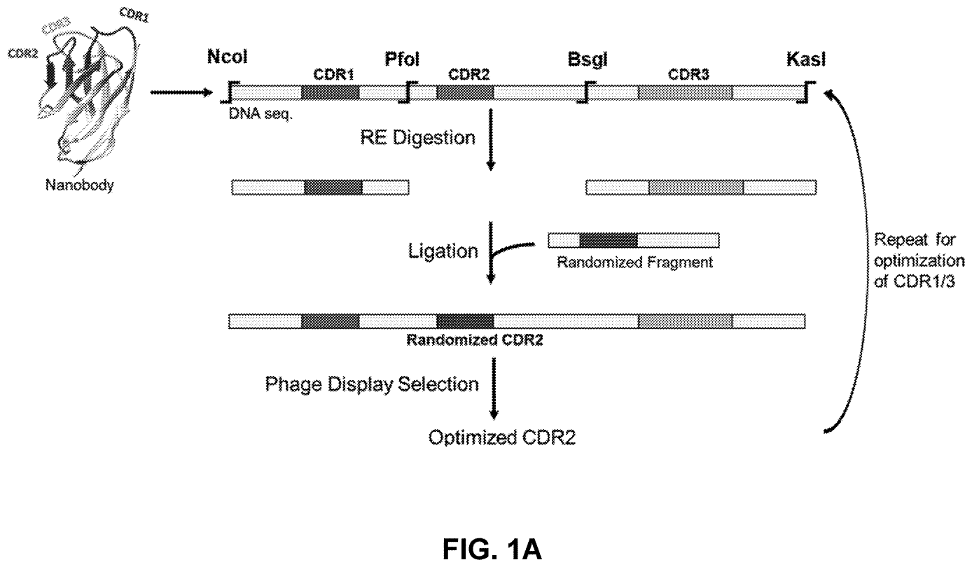

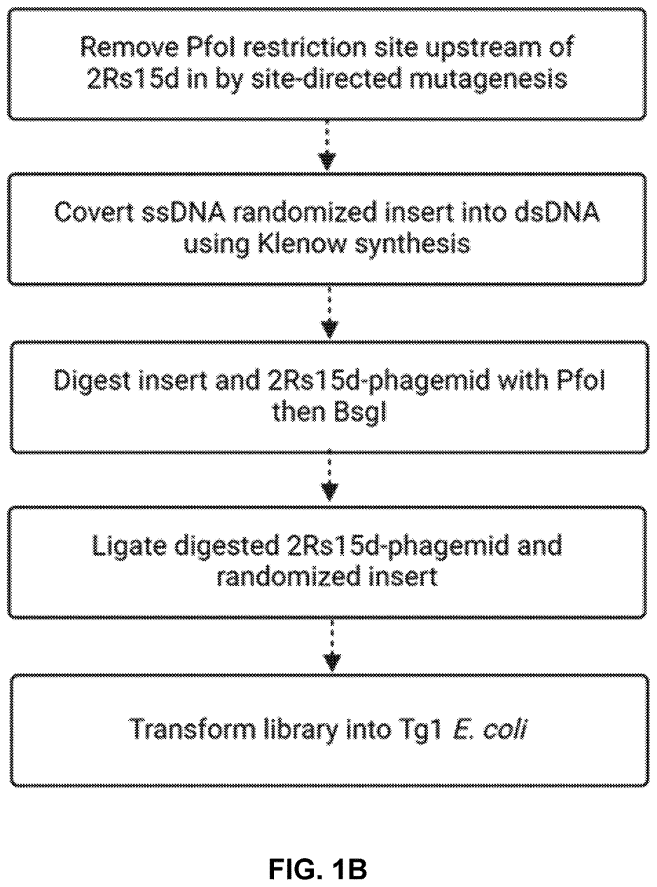

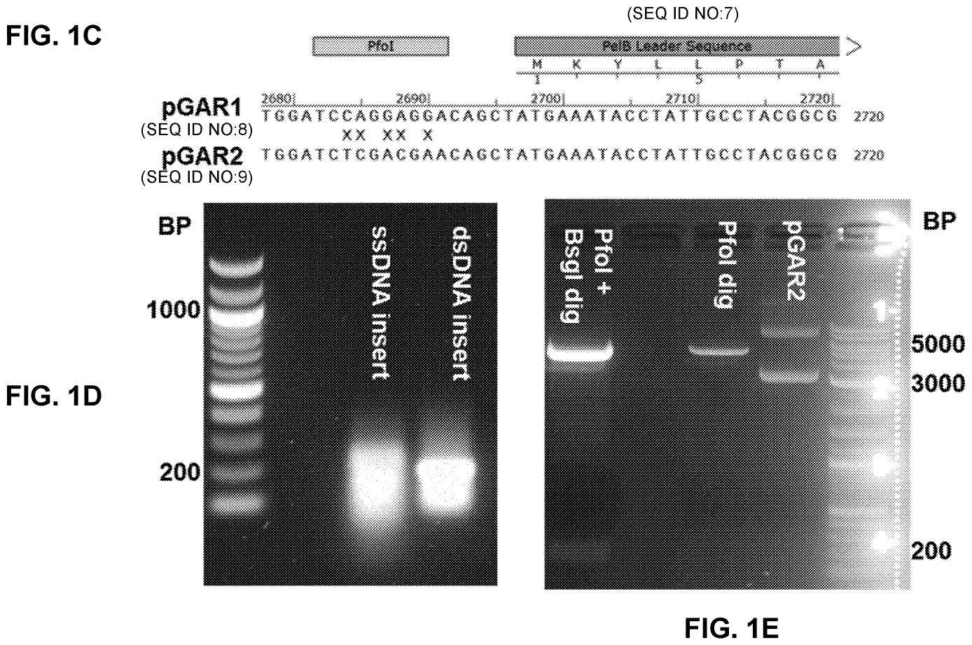

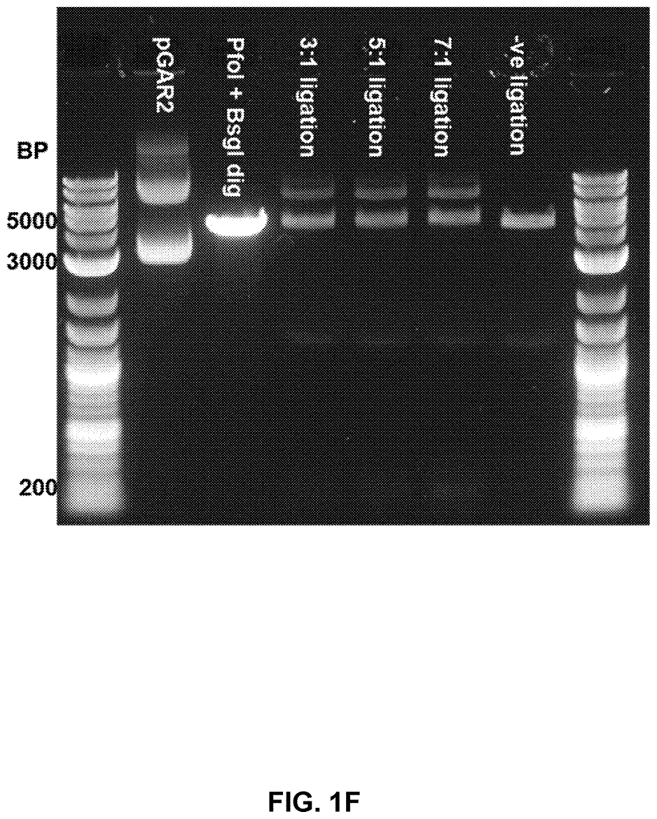

Traditional nanobody (Nb) development through naïve camelid libraries generates a diversity of 109 molecules due to sequence conservation, much lower than the theoretical diversity of fully randomized CDRs. Moreover, this strategy does not allow for modification of the Nb scaffold. In addressing this constraint, the DNA sequence of the 2rs15d phagemid has been structured to facilitate modular modification of either framework or CDR. This distinctive characteristic enables the selection for target binding and the fine-tuning of the biochemical properties of any Nb chosen from the library (FIG. 1A). Furthermore, a single synthetic library can be used to select for many targets while an immunized library is specific for a single target. Finally, a randomized CDR library can be used to generate novel Nbs for virtually any target. The generation, selection, and screening of synthetic libraries is a rapid, high-throughput process that would allow for the development of novel Nbs quicker than standard immunization and biopanning protocols.

For this selection campaign against calreticulin (CALR), we started with the randomization with CDR2 due to its lack of conserved cysteine residues involved in disulfide bonds. These cysteine residues are typically present in CDR1 and CDR3 and are critical to nanobody structure. Leaving this bond intact minimizes the effect of the randomized CDR2 on the structure and functionality of the nanobody library, increasing the likelihood of isolating a highly specific binder. The resulting nanobody library is referred to as Nanobody Library of randomized CDR2, or NaLiC2.

As outlined in FIG. 1B, the procedure to generate NaLiC2 begins abolishing the PfoI restriction site present upstream of the PelB leader sequence that guides the shuttling of the pIII-Nb fusion protein to the periplasm in E. coli. Using site directed mutagenesis, bases were substituted to read as synonymous codons. Local alignment of the DNA sequences confirmed the alteration of the restriction site in pGAR2, the phagemid used for the development of NaLiC2. Next, Klenow synthesis was used to synthesis double stranded DNA from the 200 base pair single stranded DNA insert. Following confirmation of Klenow synthesis by gel electrophoresis (FIG. 1D), visible by the DNA appearing less diffuse after synthesis, both the double stranded insert and phagemid were digested with first PfoI and then BsgI, and the successful digestion of pGAR2 was confirmed by gel electrophoresis (FIG. 1E). The single cut of pGAR2 is observed by the plasmid DNA condensing to a single band of approximately 4600 base pairs. Furthermore, the successful double digestion of PfoI and BsgI is confirmed by the 200 base pair band present in the lane. Finally, the digested insert and phagemid were ligated, and the ligation products were visible on an agarose gel (FIG. 1F) in the positive ligation reactions but not the ligation reaction ran without insert. The successfully ligated library was transformed into phage competent Tg1 E. coli.

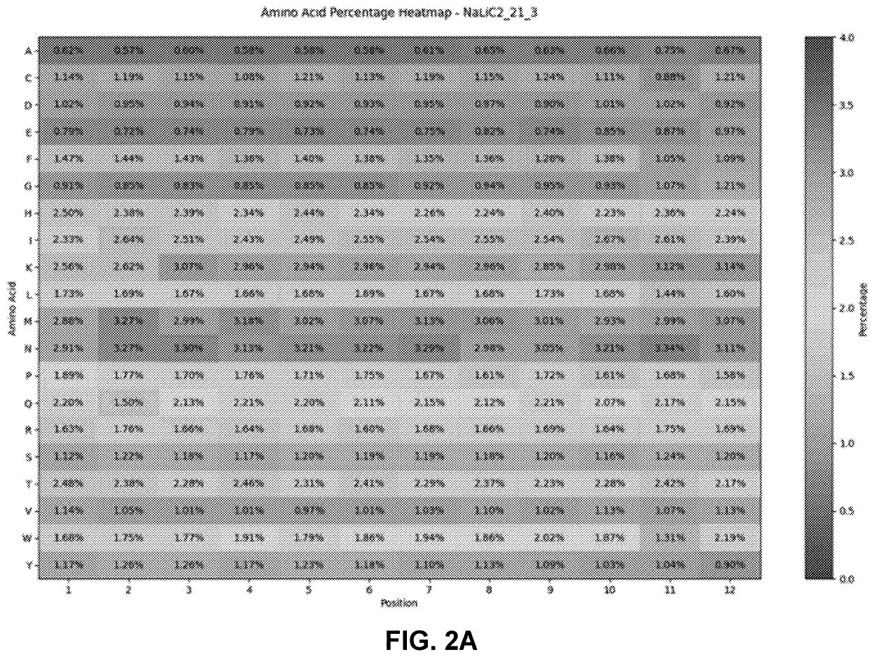

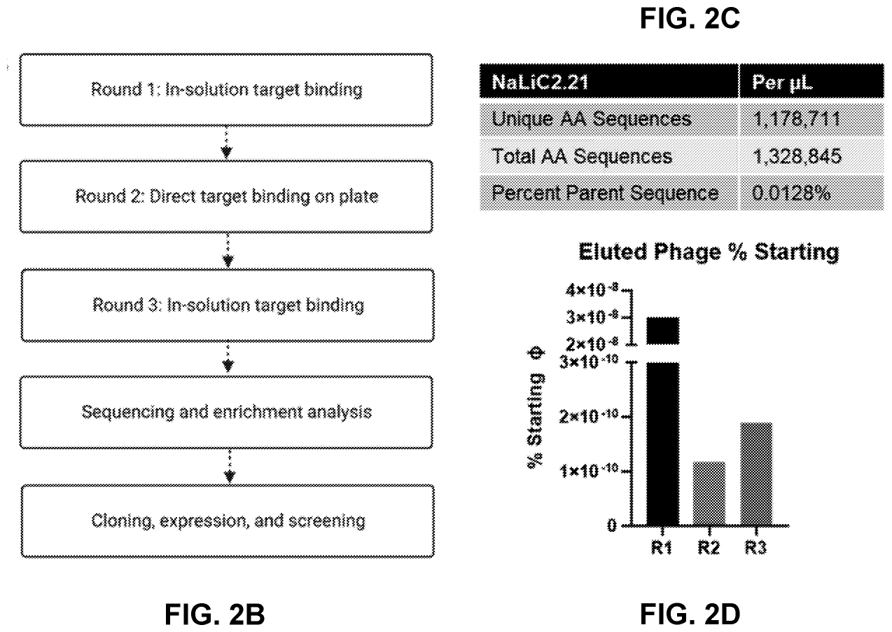

To evaluate the diversity and magnitude of the novel NaLiC2, a portion of the library was used for deep sequencing analysis of the phagemids. An in-house software was developed to identify CDR2 sequences, translate them into amino acids, and track clonal enrichment across each round of selection. Counting the occurrences of each amino acid at every position, a heatmap of the composition of CDR2 amino acid sequences was generated (FIG. 2A). The library shows a slight bias for certain groups of amino acids across all CDR2 positions. These include the nonpolar residues isoleucine and methionine; polar asparagine, glutamine, and threonine; and positively charged histidine and lysine. However, the enrichment for each of these amino acids is very low with a maximum of 3.34% over other amino acids in that position. This indicates that the library covers a broad landscape of protein sequences without a preference for particular motifs.

Of the portion of the library analyzed, 1.1×106 unique amino acid sequences per μL were identified of a total of 1.3×106 DNA sequences. 88.7% of NaLiC2 sequences are unique (FIG. 2C). Only 0.0128% of all amino acid sequences coded for the parent 2Rs15d CDR2, suggesting that the production of the NaLiC3 library was efficient. Translating these DNA sequences into amino acids, the portion of the library analyzed contained 1.1×106 unique amino acid sequences out of a total of 1.3×106 sequence. 88.7% of all amino acid sequences are unique. Confident in the diversity of NaLiC2, selections for nanobodies with affinity for CALR began.

Selections of NaLiC2 alternated between the in-solution binding of biotinylated CALR for rounds 1 and 3 and the binding of immobilized CALR in round 2 (FIG. 2B). This strategy prevented the bias of binders for epitopes unaltered or masked for biotinylation, better representing the state of CALR in vivo. The amplified phagemids following each round of selection were isolated and sequenced like the starting library to identify clones that showed enrichment across each round of selection. Calculating the eluted phage as a percentage of the number of phage used for the selection, 3.03×10-8% of the starting phage were recovered after round 1 of selection. This recovery fell 26,000-fold during round 2, indicating an enhanced stringency in the direct target binding selection technique. 1.89×10-10% of phage were recovered during round 3 of selection, an increase of 62% from round 2, suggesting an amplification of CALR specific binders.

Analyzing the sequence diversity after each round of selection, the most significant decrease in unique sequences occurs between round 1 and round 2 (FIG. 3A). This further supports the recovered phage data (FIG. 2D) that suggests that the direct target binding selection technique in round 2 is more stringent than the in-solution binding used in rounds 1 and 3. This decrease in diversity can indicate that due to clonal enrichment, there were fewer nanobodies present after 3 rounds of selection, but these clones were present in higher quantities. The next step was to determine which clones would be selected for cloning, expression, and characterization of CALR binding. The 3 most highly enriched clones in the round 3 sequencing data were coined Cal1, Cal2, and Cal3. Their CDR2 sequences are shown in FIG. 3D. Their frequency was plotted from the starting library and for each round of selection. As a control to show that Cal1-3 enrichment did not occur by random chance, the frequency of the top 4 clones from round 1 of selection were also plotted as their CDR2 sequences. While Cal1-3 frequencies grew after each round of selection, their enrichment was the greatest between rounds 2 and 3. Contrastingly, the controls from round 1 may have shown some enrichment between rounds 1 and 2 of selection, but they were not present after 3 rounds of selection. The enrichment analysis of Cal1-3 was sufficient to clone and express the nanobodies.

The CDR2 sequences for Cal1, Cal2, and Cal3 were cloned into a pET22b vector and expressed, and the resulting expression products were purified using the hexahistidine tag present at the C-terminus of the nanobody. No purification product from Cal1 or Cal2 was detected by SDS-PAGE in any of the three elutions from the NTA-nickel resin. However, a band can be seen at ~17 kDa in E1 and E2 of Cal3. While the nanobody itself is approximately 14 kDa, the C-terminal FLAG and hexahistidine tags for detection and purification bring the total molecular weight to 17.1 kDa. The E1 band intensity is reported as relative fluorescence units in FIG. 3D. While Cal1 and Cal2 had sequences corresponding to full-length nanobodies not truncated by premature stop codons, they were unable to be expressed. As such, their sequences may encode a growth advantage to phage when expressed as a pIII-Nb fusion protein. Cal3 was further characterized for its binding to CALR.

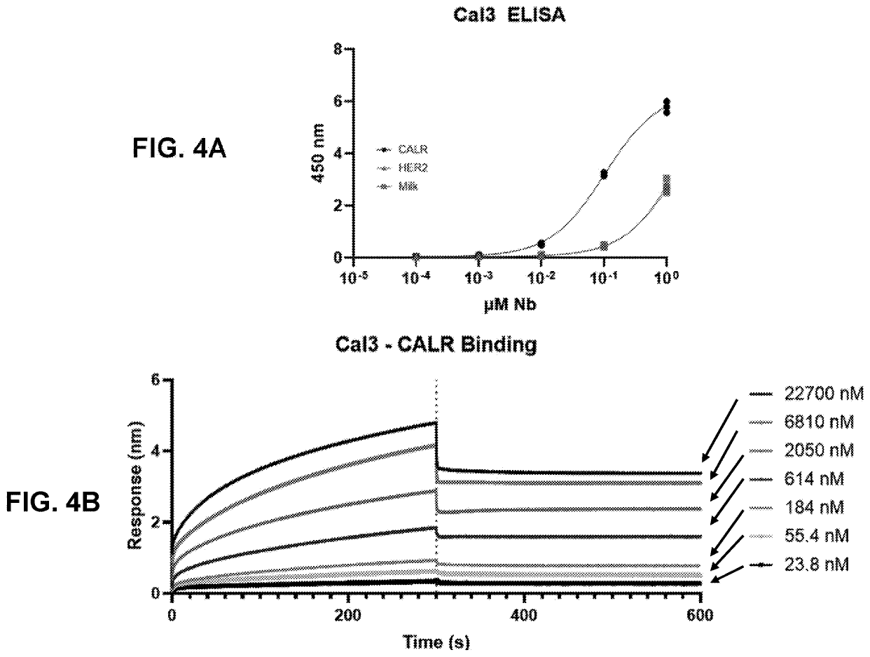

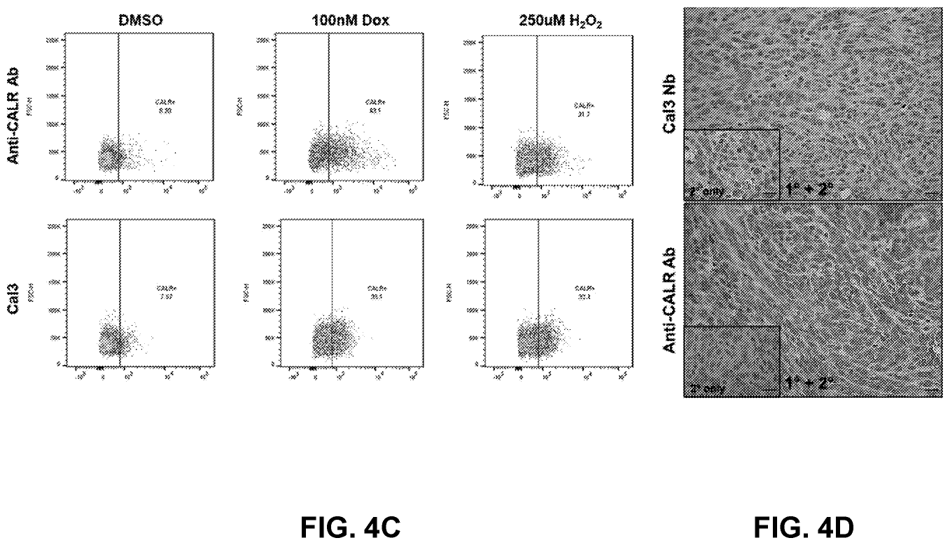

After initial screens for expression and CALR binding, it was critical to determine the affinity of Cal3 for CALR after randomization and selection of just one binding region. The apparent affinity of Cal3 for recombinant hCALR is 105 nM, determined by enzyme-linked immunosorbent assay (ELISA) (FIG. 4A). The measured affinity for HER2, the parent nanobody's original target, was similar to that of the milk block, 1.39 and 1.96 μM, respectively. To further refine the affinity of Cal3 for CALR, biolayer interferometry (BLI) was performed, and Cal3 was determined to bind CALR with an affinity of 140 nM±4.4 nM. While this affinity is 1 to 2 orders of magnitude lower than most reported nanobodies, it is a sufficient affinity for in vitro and in vivo applications.

To be useful in both research and clinical applications, Cal3 would benefit from cross reactivity with human and mouse CALR. An alignment of the protein sequences for both species shows that CALR is highly conserved between humans and mice with an identity of 393/417 amino acids (94.24%) and a similarity of 407/417 amino acids (97.60%). This conservation indicates that the epitope of Cal3 is likely maintained in both species. Additionally, one concern in using recombinant protein in phage selections is the availability of the selected nanobody's epitope in a cellular context. To address both concerns, flow cytometry was used to assess the ability to bind induced surface CALR in a murine pancreatic ductal adenocarcinoma cell line 2838c3. CALR translocation was stimulated with treatment with doxorubicin and H2O2. Under both treatment conditions, Cal3 detected surface CALR at similar frequencies as a validated anti-CALR control antibody (FIG. 4C). For further confirmation of the ability of Cal3 to bind murine cellular CALR, immunohistochemistry analysis was conducted on sectioned 2828c3 tumors. Staining with Cal3 exhibited similar patterns observed in tissues stained with the control anti-CALR antibody. These data together indicate that Cal3 is capable of binding human and mouse CALR in both recombinant and cellular formats.

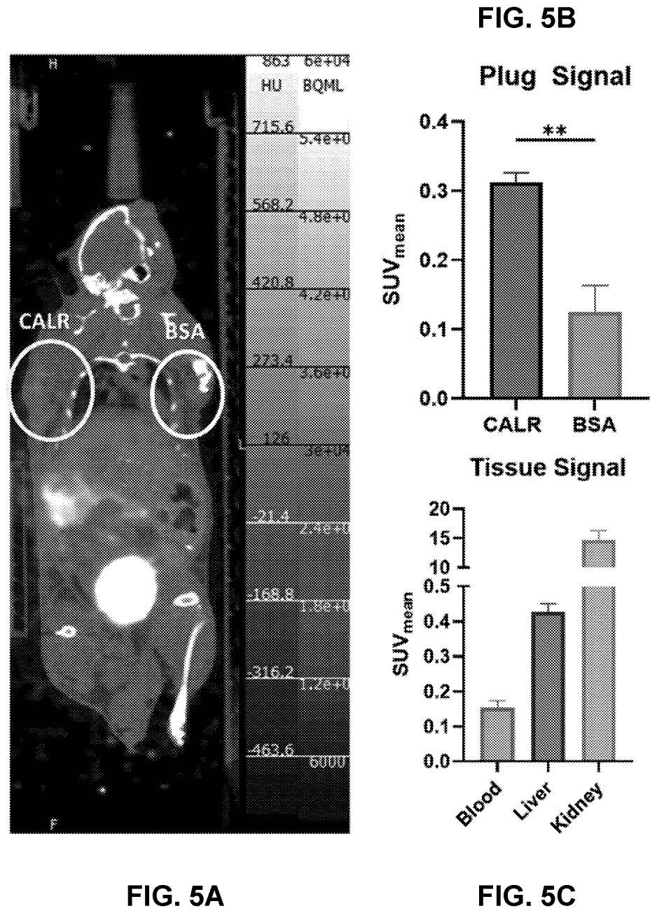

To be valuable as a theranostic nanobody, Cal3 needs to be able to detect CALR in vivo. Positron emission tomography (PET) imaging is a valuable pre-clinical and clinical tool in evaluating dynamic processes, such as the translocation of CALR from the endoplasmic reticulum to the cell surface. Additionally, PET imaging allows for the study of the whole body noninvasively and quantitatively. As such, the off-target effects of induction treatment can be evaluated with this technique. While the biological phenomenon of CALR translocation is well-established, the murine model is less developed. As such, C57/BL6 mice were subcutaneously implanted with Matrigel plugs on opposite shoulders containing either hCALR or BSA as a control protein. This recombinant protein model establishes the ability of Cal3 to bind CALR in vivo without the confounding factors found in a tumor model. After placing the plugs, mice were injected with Cal3 radiolabeled with 64-copper (64Cu), a PET radioisotope, and scanned 1 hour post injection, a timepoint typical of other nanobody-based PET tracers (FIG. 5A).

Imaging and subsequent biodistribution analysis reveals that Cal3 exhibits a distribution similar to that of other nanobody-based PET tracers. The nanobody has very high uptake in the kidneys, followed by moderate uptake in the liver and minimal uptake in other healthy tissues (FIG. 5C). There is a significantly higher uptake of 64Cu-NOTA-Cal3 detected in the CALR plugs compared to the BSA plugs, an over 2-fold increase. This data shows that Cal3 can be used as a PET imaging probe to detect CALR in vivo.

### D. Discussion

Described herein is the first synthetic nanobody for use as a PET imaging agent. To do so, a synthetic nanobody phage display library was developed, selected for novel binders for calreticulin, and validated an isolated clone both in vivo and in vitro. The generated randomized CDR2 library, NaLiC2, contained over 1.7 million unique sequences per microliter. From this library, Cal3 was isolated, a nanobody with a CDR2 specific to calreticulin with an affinity of approximately 140 nM. Cal3 was determined to be cross-reactive with both human and mouse calreticulin and capable of binding the cellular epitope presented during calreticulin translocation. Finally, Cal3 is able to detect calreticulin in vivo as a PET imaging probe.

While calreticulin was selected as a proof-of-concept target for synthetic nanobody development, it is a promising theranostic target. While a peptide specific for CALR have been described, the Cal3 is the first nanobody to be developed [22].

Finally, as described herein, synthetic nanobody development is a viable alternative to immunization for nanobodies with in vivo applications. Immune libraries remain dominant in the practice of nanobody engineering, but synthetic libraries possess many advantages over immunization [4]. Foremost is the speed at which a synthetic library can be generated and selections can be conducted. Using the modular nanobody phagemid we generated, a library can be developed in less than two weeks, and the subsequent selections could take another two weeks. This timeline is significantly condensed rather than the several months required for immunization of large mammals [4]. Even repeating this process for the affinity maturation of CDR1 and CDR3 would be faster than panning an immunized library. The modularity can also be used to select for unique properties imparted by the scaffold by introducing diversity to the framework regions rather than the CDRs [7]. NaLiC2 has the potential not only generate novel nanobodies but optimize existing nanobodies.

### E. References Related to the Present Example

1. Khodabakhsh, F., et al., Single-Domain Antibodies or Nanobodies: A Class of Next-Generation Antibodies. International Reviews of Immunology, 2018. 37(6): p. 316-322.
2. Berland, L., et al., Nanobodies for Medical Imaging: About Ready for Prime Time? Biomolecules, 2021. 11(5).
3. Pardon, E., et al., A general protocol for the generation of Nanobodies for structural biology. Nat Protoc, 2014. 9(3): p. 674-93.
4. Muyldermans, S., A guide to: generation and design of nanobodies. Febs j, 2021. 288(7): p. 2084-2102.
5. Benhar, I., Design of synthetic antibody libraries. Expert Opinion on Biological Therapy, 2007. 7(5): p. 763-779.
6. Liu, B. and D. Yang, Easily Established and Multifunctional Synthetic Nanobody Libraries as Research Tools. International Journal of Molecular Sciences, 2022. 23(3): p. 1482.
7. Valdés-Tresanco, M.S., et al., Structural Insights into the Design of Synthetic Nanobody Libraries. Molecules, 2022. 27(7): p. 2198.
8. Keyaerts, M., et al., Phase II trial of HER2-PET/CT using 68Ga-anti-HER2 VHH1 for characterization of HER2 presence in brain metastases of breast cancer patients. Annals of Oncology, 2019. 30: p. iii25-iii26.
9. Keyaerts, M., et al., Phase I Study of <sup>68</sup>Ga-HER2-Nanobody for PET/CT Assessment of HER2 Expression in Breast Carcinoma. Journal of Nuclear Medicine, 2016. 57(1): p. 27-33.
10. Vaneycken, I., et al., Preclinical screening of anti-HER2 nanobodies for molecular imaging of breast cancer. The FASEB Journal, 2011. 25(7): p. 2433-2446.
11. D'Huyvetter, M., et al., Development of 177Lu-nanobodies for radioimmunotherapy of HER2-positive breast cancer: evaluation of different bifunctional chelators. Contrast Media Mol Imaging, 2012. 7(2): p. 254-64.
12. Dekempeneer, Y., et al., Labeling of Anti-HER2 Nanobodies with Astatine-211: Optimization and the Effect of Different Coupling Reagents on Their in vivo Behavior. Molecular Pharmaceutics, 2019. 16(8): p. 3524-3533.
13. D'Huyvetter, M., et al., 131I-labeled Anti-HER2 Camelid sdAb as a Theranostic Tool in Cancer Treatment. Clinical Cancer Research, 2017. 23(21): p. 6616-6628.
14. Ducharme, M., et al., Evaluation of [89Zr]Zr-DFO-2Rs15d Nanobody for Imaging of HER2-Positive Breast Cancer. Molecular Pharmaceutics, 2023. 20(9): p. 4629-4639.
15. Debie, P., et al., Size and affinity kinetics of nanobodies influence targeting and penetration of solid tumours. Journal of Controlled Release, 2020. 317: p. 34-42.
16. Xavier, C., et al., Synthesis, Preclinical Validation, Dosimetry, and Toxicity of <sup>68</sup>Ga-NOTA-Anti-HER2 Nanobodies for iPET Imaging of HER2 Receptor Expression in Cancer. Journal of Nuclear Medicine, 2013. 54(5): p. 776-784.
17. Unterrainer, M., et al., Recent advances of PET imaging in clinical radiation oncology. Radiation Oncology, 2020. 15(1): p. 88.
18. Berger, A., How does it work? Positron emission tomography. Bmj, 2003. 326(7404): p. 1449.
19. Krysko, D. V., et al., Immunogenic cell death and DAMPs in cancer therapy. Nature Reviews Cancer, 2012. 12(12): p. 860-875.
20. Fucikova, J., et al., Detection of immunogenic cell death and its relevance for cancer therapy. Cell Death & Disease, 2020. 11(11): p. 1013.
21. Zitvogel, L., et al., Immunogenic Tumor Cell Death for Optimal Anticancer Therapy: The Calreticulin Exposure Pathway. Clinical Cancer Research, 2010. 16(12): p. 3100-3104.
22. Kim, D.-Y., et al., Imaging Calreticulin for Early Detection of Immunogenic Cell Death During Anticancer Treatment. Journal of Nuclear Medicine, 2021. 62(7): p. 956-960.
23. Consortium, T.U., UniProt: the Universal Protein Knowledgebase in 2023. Nucleic Acids Research, 2022. 51(D1): p. D523-D531.
24. Wittrup, K. D., et al., Chapter ten—Practical Theoretic Guidance for the Design of Tumor-Targeting Agents, in Methods in Enzymology, K. D. Wittrup and G.L. Verdine, Editors. 2012, Academic Press. p. 255-268.
25. Tweedle, M. F., Peptide-Targeted Diagnostics and Radiotherapeutics. Accounts of Chemical Research, 2009. 42(7): p. 958-968.
26. Laura Teodori, Sarah K. Ochoa, Marjan Omer, Veronica L. Andersen, Pernille Bech, Junyi Su, Jessica Bridoux, Jesper S. Nielsen, Mathias B. Bertelsen, Sophie Hernot, Kurt V. Gothelf, Jørgen Kjems, Plug-and-play nucleic acid-mediated multimerization of biparatopic nanobodies for molecular imaging, Molecular Therapy—Nucleic Acids, Volume 35, Issue 3, 2024, 102305, ISSN 2162-2531, https://doi.org/10.1016/j.omtn.2024.102305.

Informal Sequence Listing

| | | SEQ |
|---|---|---|
| | | ID |
| Description | SEQUENCE (5′→3′; N′→C′) | NO: |
| Upstream | TATGACCATGATTACGCCAAGC | 1 |
| primer of | | |
| nanobody | | |
| sequence | | |
| CDR2-flanking | TATCGGCAGAGTCCTGGAAGAGAG | 2 |
| primer 1 | | |
| CDR2-flanking | GTTTCCAAGTTGTAACACACTGCAC | 3 |
| primer 2 | | |
| Cal1 CDR2 | STHVSALLYSKG | 4 |
| Cal2 CDR2 | GCLAVCPHRKT | 5 |
| Cal3 CDR2 | SVGDCMAGSSPN | 6 |
| PelB leader | MKYLLPTA | 7 |
| sequence | | |
| pGAR1 | TGGATCCAGGAGGACAGCTATGAAATACCTATTGCCTA | 8 |
| sequence | CGGCG | |
| pGAR2 | TGGATCTCGACGAACAGCTATGAAATACCTATTGCCTAC | 9 |
| sequence | GGCG | |
| Round 1 Top 1 | EHELIITIAYQD | 10 |
| 1 | | |
| Round 1 Top 1 | MPHLTNSLTIST | 11 |
| 2 | | |
| Round 1 Top 1 | YVGRVDPNGLNS | 12 |
| 3 | | |
| Round 1 Top 1 | IGAMGGEATCTR | 13 |
| 4 | | |
| Embodiment of | GYIFNSCG | 14 |
| Nanobody | | |
| CDR1 | | |
| Embodiment of | VCYNLETY | 15 |
| Nanobody | | |
| CDR3 | | |
| FLAG | DYKDDDDK | 16 |
| polyhistidine | HHHHHH | 17 |
| hemagglutinin | YPYDVPDYA | 18 |
| Chain B, | QVQLQESGGGSVQAGGSLKLTCAASGYIFNSCGMGWYR | 19 |
| Nanobody | QSPGRERELVSRISGDGDTWHKESVKGRFTISQDNVKKT | |
| 2Rs15d | LYLQMNSLKPEDTAVYFCAVCYNLETYWGQGTQVTVSS | |
| (CDR 1, 2, and | | |
| 3 underlined in | | |
| order of 1⇒3 | | |
| from N′ to C′) | | |
| Full-length Cal1 | QVQLQESGGGSVQAGGSLKLTCAASGYIFNSCGMGWYR | 20 |
| (CDR2 | QSPGRERELVSSTHVSALLYSKGRFTISQDNVKKTLYLQM | |
| underlined, | NSLKPEDTAVYFCAVCYNLETYWGQGTQVTVSS | |
| SEQ ID NO: 4) | | |
| Full-length Cal2 | QVQLQESGGGSVQAGGSLKLTCAASGYIFNSCGMGWYR | 21 |
| (CDR2 | QSPGRERELVSSGCLAVCPHRKTRFTISQDNVKKTLYLQ | |
| underlined, | MNSLKPEDTAVYFCAVCYNLETYWGQGTQVTVSS | |
| SEQ ID NO: 5) | | |
| Full-length Cal3 | QVQLQESGGGSVQAGGSLKLTCAASGYIFNSCGMGWYR | 22 |
| (CDR2 | QSPGRERELVSSVGDCMAGSSPNRFTISQDNVKKTLYLQ | |
| underlined, | MNSLKPEDTAVYFCAVCYNLETYWGQGTQVTVSS | |
| SEQ ID NO: 6) | | |

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

At least the following is claimed:

1. A calreticulin-targeting nanobody, comprising a heavy chain variable region comprising:
(a) a VHCDR1 polypeptide comprising an amino acid sequence having at least 90% sequence identity to SEQ ID NO:14;
(b) a VHCDR2 polypeptide comprising an amino acid sequence having at least 90% sequence identity to any one of SEQ ID NOs:4-6 or 10-13; and
(c) a VHCDR3 polypeptide comprising an amino acid sequence having at least 90% sequence identity to SEQ ID NO:15.

2. The calreticulin-targeting nanobody of claim 1, wherein
(a) the VHCDR1 polypeptide comprises an amino acid sequence having at least 95% sequence identity to SEQ ID NO:14;
(b) the VHCDR2 polypeptide comprises an amino acid sequence having at least 95% sequence identity to any one of SEQ ID NOs:4-6 or 10-13; and
(c) the VHCDR3 polypeptide comprises an amino acid sequence having at least 95% sequence identity to SEQ ID NO:15.

3. The calreticulin-targeting nanobody of claim 1, wherein
(a) the VHCDR1 polypeptide comprises SEQ ID NO:14;
(b) the VHCDR2 polypeptide comprises any one of SEQ ID NOs:4-6 or 10-13; and
(c) the VHCDR3 polypeptide comprises SEQ ID NO:15.

4. The calreticulin-targeting nanobody of claim 1, wherein the heavy chain variable region comprises a polypeptide having at least 95% sequence identity to any one of SEQ ID NOs:20-22.

5. A calreticulin-targeting nanobody according to Formula 1 below (N terminus to C terminus):
A-B-X Formula 1
wherein A is one or more paramagnetic ions, one or more x-ray imaging agents, one or more fluorophores, or one or more radioisotopes, individually in or in any combination of any thereof;
wherein B is an optional linker; and
wherein X comprises an calreticulin-targeting nanobody, comprising a heavy chain variable region comprising:
(a) a VHCDR1 polypeptide comprising an amino acid sequence having at least 90% sequence identity to SEQ ID NO:14;
(b) a VHCDR2 polypeptide comprising an amino acid sequence having at least 90% sequence identity to any one of SEQ ID NOs:4-6 or 10-13; and
(c) a VHCDR3 polypeptide comprising an amino acid sequence having at least 90% sequence identity to SEQ ID NO:15.

6. The calreticulin-targeting nanobody of claim 5, wherein
(a) the VHCDR1 polypeptide comprises SEQ ID NO:14;
(b) the VHCDR2 polypeptide comprises any one of SEQ ID NOs:4-6 or 10-13; and
(c) the VHCDR3 polypeptide comprises SEQ ID NO:15.

7. The calreticulin-targeting nanobody of claim 5, wherein the heavy chain variable region comprises a polypeptide having at least 95% sequence identity to any one of SEQ ID NOs:20-22.

8. The calreticulin-targeting nanobody of claim 5, wherein the one or more fluorophores comprise a near-infrared (NIR) cyanine dye, a hemicyanine dye, a coumarin-based dye, a fluorescein-based dye, a rhodamine-based dye, a BODIPY-Based Dye, a BODIPY-Based NIR Dye, a Xanthene-Based dye, a Xanthene-Based NIR Dye, a cyanine-based dyes, a porphyrin dye, a phthalocyanine dye, or a squaraine dye, individually or in any combination of any thereof.

9. The calreticulin-targeting nanobody of claim 5, wherein the radioisotope comprises one or more of ³H, ¹¹C, ¹⁴C, ¹⁸F, ³²P, ³⁵S, ³⁶Cl, ⁵¹Cr, ⁵²Fe, ⁵⁷Co, ⁵⁸Co, ⁵⁹Fe, ⁶⁴Cu, ⁶⁷Cu, ⁶⁷Ga, ⁶⁸Ga, ⁷⁵Se, ⁷⁶Br, ⁷⁷Br, Zr⁸⁹, ⁹⁰Y, ^{99m}Tc, ¹¹¹In, ¹²³I, ¹²⁴I, ¹²⁵I, ¹³¹I, ¹⁵²Eu, ¹⁵³Sm, ¹⁶⁶Ho, ¹⁷⁷Lu, ¹⁸⁶Re, ¹⁸⁸Re, ²⁰¹Tl, ²⁰³Pb, ²¹⁰At, ²¹¹At, ²¹²Bi, ²¹³Bi, or ²²⁵Ac, individually or in any combination of any thereof.

10. The calreticulin-targeting nanobody of claim 5, wherein A further comprises a chelating agent Ch.

11. The calreticulin-targeting nanobody of claim 10, wherein Ch comprises one or more of 1,4,7-triazacyclononanetriacetic acid (NOTA), 1,4,7,10-tetraazacyclododecane-1,4,7,10-tetraacetic acid (DOTA), 1,4,7-triazacyclononane-1-glutaric acid-4,7-diacetic acid (NODAGA), ethylene diamine tetra-acetic acid (EDTA) diethylene triaminepentaacetic acid (DTPA), cyclohexyl-1,2-diaminetetraacetic acid (CDTA), ethyleneglycol-O,O′-bis(2-aminoethyl)-N,N,N′,N′-tetraacetic acid (EGTA), N,N-bis(hydroxybenzyl)-ethylenediamine-N,N′-diacetic acid (HBED), triethylene tetramine hexaacetic acid (TTHA), hydroxyethyidiamine triacetic acid (HEDTA), 1,4,8,11-tetraazacyclotetradecane-N,N′,N″,N′″-tetraacetic acid (TETA), TCMC, or DFO, individually or in any combination of any thereof.

12. The calreticulin-targeting nanobody of claim 5, wherein B comprises one or more of one or more alkylene groups, one or more amine groups, one or more amide groups, one or more alkyleneoxy groups, one or more thiol groups, one or more carbohydrate groups, or any combination thereof.

13. A method of using a calreticulin-targeting nanobody, comprising:
inducing calreticulin (CALR) translocation in a plurality of cells in a biological sample;
contacting the sample with a composition comprising a nanobody of claim 1;
incubating the contacted sample at a temperature for a period of time; and
imaging the incubated sample to determine a nanobody level.

14. The method of claim 13, wherein the sample comprises a tumor, a tumor biopsy or suspected tumor in or from a subject.

15. The method of claim 14, wherein the tumor, tumor biopsy, or suspected tumor comprises an adenocarcinoma, an esophageal cancer, a gastric cancer, a small bowel cancer, a colon cancer, a rectal cancer, an anal cancer, a lunch cancer, a mesothelioma, a peritoneal cancer, an ovarian cancer, a uterine cancer, a breast cancer, a renal cancer, a urothelial cancer, a prostate cancer, a hepatoceullar carcinoma, a cholangiocarcinoma, a lymphoma, a leukemia, a liposarcoma, a, rhabdosarcoma, a fibrosarcoma, an angiosarcoma, a chondrosarcoma, a thyroid cancer, an oral cancer, a head and neck cancer, or a glioblastoma, individually or in any combination of any thereof.

16. The method of claim 13, wherein inducing translocation is performed by administering an inducing agent.

17. The method of claim 16, wherein the inducing agent comprises one or more of an anthracycline, gemcitabine, 5-FU, dantrolene, an oxidizing agent, or a platinium-based inducing agent.

18. The method of claim 13, wherein the imaging is positive emission tomography (PET) scanning.

19. A composition, comprising:
a calreticulin-targeting nanobody of claim 1; and
a carrier.

20. A kit, comprising:
one or more nanobodies of claim 1; and
Instructions for use.


SEQUENCE LISTING (22 sequences)

SEQ ID NO: 1 - DNA, 22 bp, synthetic construct
  source 1..22: mol_type = other DNA
tatgaccatgattacgccaagc

SEQ ID NO: 2 - DNA, 24 bp, synthetic construct
  source 1..24: mol_type = other DNA
tatcggcagagtcctggaagagag

SEQ ID NO: 3 - DNA, 25 bp, synthetic construct
  source 1..25: mol_type = other DNA
gtttccaagttgtaacacactgcac

SEQ ID NO: 4 - protein, 12 aa, synthetic construct
STHVSALLYSKG

SEQ ID NO: 5 - protein, 11 aa, synthetic construct
GCLAVCPHRKT

SEQ ID NO: 6 - protein, 12 aa, synthetic construct
SVGDCMAGSSPN

SEQ ID NO: 7 - protein, 8 aa, synthetic construct
MKYLLPTA

SEQ ID NO: 8 - protein, 43 aa, synthetic construct
TGGATCCAGGAGGACAGCTATGAAATACCTATTGCCTACGGCG

SEQ ID NO: 9 - DNA, 43 bp, synthetic construct
  source 1..43: mol_type = other DNA
tggatctcgacgaacagctatgaaatacctattgcctacggcg

SEQ ID NO: 10 - protein, 12 aa, synthetic construct
EHELIITIAYQD

SEQ ID NO: 11 - protein, 12 aa, synthetic construct
MPHLTNSLTIST

SEQ ID NO: 12 - protein, 12 aa, synthetic construct
YVGRVDPNGLNS

SEQ ID NO: 13 - protein, 12 aa, synthetic construct
IGAMGGEATCTR

SEQ ID NO: 14 - protein, 8 aa, synthetic construct
GYIFNSCG

SEQ ID NO: 15 - protein, 8 aa, synthetic construct
VCYNLETY

SEQ ID NO: 16 - protein, 8 aa, synthetic construct
DYKDDDDK

SEQ ID NO: 17 - protein, 6 aa, synthetic construct
HHHHHH

SEQ ID NO: 18 - protein, 9 aa, synthetic construct
YPYDVPDYA

SEQ ID NO: 19 - protein, 115 aa, synthetic construct
QVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSS

SEQ ID NO: 20 - protein, 111 aa, synthetic construct
QVQLQESGGGSVQAGGSLKLTCAASGYIFNSCGMGWYRQSPGRERELVSSTHVSALLYSKGRFTISQDNVKKTLYLQMNSLKPEDTAVYFCAVCYNLETYWGQGTQVTVSS

SEQ ID NO: 21 - protein, 111 aa, synthetic construct
QVQLQESGGGSVQAGGSLKLTCAASGYIFNSCGMGWYRQSPGRERELVSSGCLAVCPHRKTRFTISQDNVKKTLYLQMNSLKPEDTAVYFCAVCYNLETYWGQGTQVTVSS

SEQ ID NO: 22 - protein, 111 aa, synthetic construct
QVQLQESGGGSVQAGGSLKLTCAASGYIFNSCGMGWYRQSPGRERELVSSVGDCMAGSSPNRFTISQDNVKKTLYLQMNSLKPEDTAVYFCAVCYNLETYWGQGTQVTVSS